(19) United States
(12) Patent Application Publication
(10) Pub. No.: US 2026/0224475 A1
(43) Pub. Date: Aug. 6, 2026

(54) MULTI-COMPONENT INTRAVAGINAL RING

(71) Applicants: THE POPULATION COUNCIL, INC., New York, NY (US); THE QUEEN'S UNIVERSITY OF BELFAST, Belfast (GB); CORNELL UNIVERSITY, Ithaca, NY (US)

(72) Inventors: Lisa HADDAD, New York, NY (US); Natalia TELESHOVA, New York, NY (US); Narender KUMAR, New York, NY (US); Dolores J. LAMB, Ithaca, NY (US); Peter BOYD, Belfast (GB); Karl MALCOLM, Belfast (GB)

(21) Appl. No.: 19/147,221

(22) PCT Filed: Jan. 15, 2024
(86) PCT No.: PCT/US2024/011541

§ 371 Date: Jul. 10, 2025

Related U.S. Application Data
(60) Provisional application No. 63/479,848, filed on Jan. 13, 2023.

(51) Int. Cl. A61K 9/00; A61F 6/08; A61K 31/19; A61K 31/357; A61K 33/30; A61K 33/34; A61P 15/18; A61P 31/04; A61P 31/18; A61P 31/22

(52) U.S. Cl. CPC A61K 9/0036; A61F 6/08; A61K 31/19; A61K 31/357; A61K 33/30; A61K 33/34; A61P 15/18; A61P 31/04; A61P 31/18; A61P 31/22

(57) ABSTRACT

The present disclosure relates to a multi-component intravaginal ring (IVR) for improved release of non-hormonal agents useful for multipurpose prevention technology (MPT) that simultaneously prevents unintended pregnancy, and prevents or treats sexually transmitted infections (STIs) while at the same time optimizing vaginal health.

### CROSS-REFERENCE TO RELATED APPLICATION

The present application claims the priority from U.S. Provisional Patent Application No. 63/479,848 filed on Jan. 13, 2023, the disclosure of which is incorporated by reference herein.

This invention was made with government support under P50HD106793 awarded by the National Institutes of Health (NIH). The government has certain rights in the invention.

### FIELD OF THE INVENTION

The present disclosure relates to a multi-component intravaginal ring (IVR) for improved release of non-hormonal agents useful as a multipurpose prevention technology (MPT) product that simultaneously prevents unintended pregnancy, prevents and/or treats one or more sexually transmitted infections (STIs), and promotes vaginal health.

### BACKGROUND OF THE INVENTION

Unprotected sex can result in unintended pregnancy and STIs (including HIV-1, HSV-2, and others), which represent major reproductive and sexual health problems for women worldwide.

Although hormonal methods such as oral hormonal contraceptives, hormonal vaginal ring and hormonal intrauterine devices (IUDs) are highly effective in preventing pregnancy, they do not protect against STIs. Further, long-term use of hormonal products can increase risks of adverse events such as blood clots and may be poorly tolerated or contraindicated for use in some individuals.

Currently, the only non-hormonal options for preventing pregnancy are copper IUDs and various pericoital devices/products (including male and female condoms, cervical diaphragms/caps, vaginal sponges, vaginal films, and the recently approved acid buffering Phexxi® gel). However, copper IUDs are required to insert by a health care provider, and pericoital approaches give low typical use effectiveness.

Women overwhelmingly prefer and are more likely to use a MPT product that simultaneously prevents unintended pregnancy and STIs over products that prevent only pregnancy or only STIs. To date, condoms remain the only approved MPTs, but they suffer from inconsistent use and typical use failure rate of 13%.

Drug-releasing IVRs are flexible torus-shaped device mostly fabricated from medical grade thermosetting materials (such as silicone elastomers) or thermoplastic materials (such as ethylene-vinyl acetate copolymer) and offer sustained or controlled release of therapeutic agents to the vagina for clinical benefit. Traditionally, IVRs (i) provide a convenient formulation option for administration of drugs having certain physicochemical characteristics, (ii) offer relatively low variability in drug release rate, (iii) minimize systemic absorption of drugs, (iv) avoid hepatic first-pass metabolism of the drug substance (where drug absorption does occur), and (v) offer improved user adherence and acceptability/satisfaction compared with other vaginal dosage forms.

Permeation-controlled release of substances from current marketed IVRs requires the incorporated substance(s) to first dissolve in and then diffuse through the polymer material used in construction of the device. To date, efforts to advance IVRs for the release of non-hormonal agents have been limited, due to the poor solubility and permeability of the non-hormonal agents (which are normally water-soluble or hydrophilic) in the hydrophobic polymers commonly used to manufacture IVRs and/or the relative lack of potency of non-hormonal agents.

Efforts to overcome these limitations to extend vaginal ring technology to a wider range of actives have included: (i) rings made from biosoluble acacia gum or non-biodegradable hydrogel excipients; (ii) rings manufactured using alternative polymers, such as hydrophilic/water-swellable polyurethanes; (iii) rings containing or made from components having relatively high concentrations of hydrophilic active agents and excipients; and (iv) rings containing one or more discrete drug-loaded rods or pods that control release of the drug active(s).

For example, Ovaprene® is the leading non-hormonal IVR currently in development. Designed to provide contraception over multiple weeks, the device provides its contraceptive activity in two ways—it contains a semi-permeable polymer mesh barrier that physically blocks sperm from entering the cervical canal, and it releases ferrous gluconate from the main ring body that acts locally to impede sperm motility.

Brij et al. reported development of an IVR composed of nanoporous poly(diol citrate) elastomer hydrogel for a sustained release of nonhormonal contraceptives and anti-HIV agents, namely ferrous gluconate, L-ascorbic acid, and mixtures of polyamino-polycarboxylic acid (Ampholines) or Poly L-Glutamic Acid (PLGA):sodium bicarbonate buffer (Development of a Nanoporous Elastomere Intra-Vaginal Ring (IVR) for the Sustained Release of Non-Hormonal Contraceptives, J. Pharm. Drug Deliv. Res. 2012, 1:1).

Inevitably, many of these newer IVR designs require complex multi-step manufacturing processes that are difficult and expensive to scale and manufacture. Using high concentration of an active agent may cause cytotoxic effects on the vaginal epithelial tissue. Incorporation of multiple agents into the same compartment within a ring can lead to drug-drug interactions, with potential implications for drug stability and drug release. Further, combining different drugs in the same IVR poses challenges due to the different solubilities and target release rates of different drugs.

With over 1 million new daily cases of STIs globally, new MPT methods need to provide broad-spectrum STI coverage, as a narrow STI prevention/treatment scope could lead to behavioral risk-compensation where protection from or treatment of one pathogen can increase the risk of acquiring others.

There is a need in developing new IVRs that: (i) offer sustained/controlled release of non-hormonal agents, (ii) are practical to use, (iii) are highly efficacious in preventing unintended pregnancy and preventing/treating one or more STIs, and (iv) can be easily manufactured using conventional and scalable processes.

### SUMMARY OF THE INVENTION

In one aspect, the present disclosure is directed to an IVR for intravaginally administering a therapeutically effective amount of a non-hormonal active agent or a combination thereof to a female subject.

In one aspect, the present disclosure is directed to a method of making an IVR for intravaginally administering an active ingredient or a combination thereof to a female subject.

In one aspect, the present disclosure is directed to a method of preventing unintended pregnancy, and preventing or treating a disease caused by bacterial and viral infection by applying an IVR intravaginally for the purpose of administering a pharmaceutically effective amount of an active agent or a combination thereof to a female subject.

In one embodiment, the IVR may comprise a therapeutically effective amount of a non-hormonal active agent dispersed in an elastomer.

In one embodiment, the non-hormonal active agent may comprise at least one selected from a copper component, a zinc component or a lactide component.

In one embodiment, the non-hormonal active agent may comprise a lactide component, and at least one selected from a copper component, or a zinc component.

In one embodiment, the non-hormonal active agent may comprise a lactide component, a copper component, and a zinc component.

In one embodiment, the copper component may be metallic copper, copper oxide, a copper salt or a copper ion-ligand complex.

In one embodiment, the zinc component may be metallic zinc, zinc oxide or a zinc salt.

In one embodiment, the lactide component may be D-lactide, L-lactide, DL-lactide or lactic acid.

In one embodiment, the elastomer may be selected from silicone, polyethylene vinyl acetate copolymer (EVA), styrene-butadiene-styrene block copolymer, polyphosphazene, poly(isoprene), poly(isobutylene), polybutadiene, polyurethane, a nitrile rubber, a neoprene rubber, or a combination thereof.

In one embodiment, the elastomer may be included in an amount of about 50% to about 99% by a total weight of the IVR.

In one embodiment, the IVR may be a form of a matrix, and the elastomer is silicone.

In one embodiment, the IVR is in a form of an exposed-core, and the elastomer is polyethylene vinyl acetate or polyurethane.

In one embodiment, the copper salt may be anhydrous copper sulfate or copper sulfate hydrate.

In one embodiment, the zinc salt may be zinc acetate, zinc formate, zinc lactate, zinc chloride, zinc sulfate, zinc iodide, zinc citrate, or zinc orotate, each of which is in an anhydrous or a hydrate form.

In one embodiment, the non-hormonal active agent may be included in an amount of about 5 wt % to about 50 wt %, based on a total amount of the IVR.

In one embodiment, the IVR may have copper ions, zinc ions and lactic acid released in a molar ratio of 1:1:1 to 1:6:6 after an initial first day period.

In one embodiment, the IVR may have a release of copper ions at a rate of 2 mg/day to 31 mg/day during a 30-day period of use.

In one embodiment, the IVR may have a release of zinc ions at a rate of 1 mg/day to 17 mg/day during a 30-day period of use.

In one embodiment, the IVR may have a release of lactic acid at a rate of 12 mg/day to 105 mg/day during a 30-day period of use.

### BRIEF DESCRIPTION OF DRAWINGS

FIG. 1 illustrates difference in zinc salts for 50% motility reduction with 95% confidence interval (CI).

FIG. 2 illustrates sperm motility dose-response for single API.

FIG. 3 illustrates sperm mucus penetration results for single API and CS-ZL-LA (CSL) combination.

FIG. 4 illustrates synergy plotted against efficacy (stated as the fraction affected (Fa) for 2-API and 3-API combos.

FIG. 5 illustrates motility inhibition for CS-ZL-LA combination.

FIG. 6 illustrates percentage of motile sperm at different experimental stages for API (CS-ZL-LA combination) and SVF control.

FIG. 7 illustrates that percentage of sperm undergoing acrosome exocytosis after 3-hour incubation in capacitating conditions.

FIG. 8 illustrates percentage of dead or damaged pattern of Sybr14/Propidium Iodide staining after the wash step or after 3-hour incubation in capacitating conditions.

FIG. 9 illustrates percentage of sperm exhibiting CTB:488 staining patterns associated with increased membrane fluidity when incubated for 3 hours for untreated control, SVF control and API (CS-ZL-LA combination) in capacitating (5 mM 2OHCD) and non-capacitating conditions.

FIG. 10 illustrates anti-HIV-1_{BaL} activity of APIs, alone and in combination.

FIG. 11 illustrates anti-HIV-1BaL activity of APIs in the absence/presence of biological fluids and at concentrations inhibiting sperm motility.

FIG. 12 illustrates anti-HSV-2 activity of APIs, alone and in combination.

FIG. 13 illustrates anti-HSV-2 activity of APIs in the absence/presence of biological fluids alone and at concentrations inhibiting sperm motility.

FIG. 14 illustrates API's inhibitory activity against N. gonorrhoeae.

FIG. 15 illustrates that API's bactericidal activity against C. trachomatis in the presence of biological fluids.

FIG. 16 illustrates daily and cumulative release of lactide from IVRs.

FIG. 17 illustrates daily and cumulative release of lactic acid from IVRs.

FIG. 18 illustrates daily and cumulative release of copper ion from IVRs.

FIG. 19 illustrates daily and cumulative release of zinc ion from IVRs.

FIG. 20 illustrates effect of APIs on pH.

FIG. 21 illustrates pH of the release medium of the APIs during the 30-day in vitro release study.

FIG. 22 illustrates viability of the ectocervical explants following single exposure to APIs.

FIG. 23 illustrates histological evaluation of ectocervical explants following single exposure to APIs.

FIG. 24 illustrates inflammatory cytokines concentrations following single exposure of ectocervical explants to APIs.

FIG. 25 illustrates viability and TEER in VEC-100 tissue following single exposure to APIs.

FIG. 26 illustrates viability and TEER in VEC-100 tissue following repeated exposure of APIs.

### DETAILED DESCRIPTION

Before describing at least one embodiment of the invention in detail, it is to be understood that the invention is not limited in its application to the details set forth in the following description or illustrated by the examples. The invention is capable of other embodiments or of being practiced or carried out in various ways. It is therefore to be understood that numerous modifications may be made to the illustrative embodiments and that other arrangements may be devised without departing from the spirit and scope of the present disclosure. It is also to be understood that the phrasing and terminology employed herein is for the purpose of description and should not be regarded as limiting.

All measurements made herein are at room temperature (25° C.±5° C.) and normal pressure unless otherwise designated. All temperatures used herein are in degrees Celsius unless specified otherwise.

The present disclosure can comprise (open ended) or consist essentially of the components as well as other ingredients, agents or elements described herein. Unless otherwise indicated, the term “substance,” “component,” “ingredient”, “active” or “agent” used herein is exchangeable. As used herein, the term “comprising” means having the elements recited, or their equivalent in structure or function, plus any other element or elements which are not recited. The terms “having” and “including” used herein are also to be construed as open ended unless the context suggests otherwise. The terms “comprising,” “having” and “including” encompass the terms “consisting of” and “consisting essentially of.” The term “consisting essentially of” herein means that the composition or method may include additional ingredients and/or steps, but only if the additional ingredients and/or steps do not materially alter the characteristics of the claimed composition or method.

All ranges recited herein include the endpoints, and those falling between. Terms such as “about,” “generally,” “substantially” and the like are to be construed as modifying a term or value such that it is not an absolute, but does not read on the prior art. Such terms are defined by the circumstances and the terms that they modify as those are understood by one of ordinary skill in the art. This includes, at very least, the degree of expected experimental error, technique error, and instrument error for a given technique used to measure a value. Unless otherwise indicated, the term “about” as used herein includes all values in a range of the specific value indicated ±10%.

Unless otherwise indicated, as used herein, the terms “a” and “an” include the plural, such that, e.g., “a component” can mean at least one component, as well as a plurality of components including but not limited to components of different types.

Where used herein, the term “and/or” when used in a list of two or more items means that any one of the listed characteristics can be present, or any combination of two or more of the listed characteristics can be present. For example, if a composition is described as comprising agents A, B, and/or C, the composition can comprise A alone; B alone; C alone; A and B in combination; A and C in combination; B and C in combination; or A, B, and C in combination.

As used herein, the term “contraceptive” refers to an active agent or combination thereof administrated to prevent or reduce the likelihood of conception or pregnancy.

As used herein, the term “antimicrobial” refers to an active agent or combination thereof that is capable of inhibiting or destroying the growth of a microbial organism. Antimicrobial agents include, but are not limited to, antibacterial, antifungal, antiprotozal, and antiviral agents.

As used herein, the term “intravaginal ring,” “vaginal ring”, “ring” or “IVR” refers to a torus-shaped polymeric delivery device, which is designed to be inserted into the vagina of a female subject to provide administration (e.g., controlled release) of an active agent to the vagina over an extended period of time.

As used herein, the term “matrix ring” or “matrix-type ring” refers to an IVR in which an active agent or a combination thereof, optionally with other excipients, are homogenously distributed throughout the elastomeric ring. Matrix rings are typically manufactured by injection molding or extrusion of active agent(s)-containing mixture, leading to a uniform distribution of the active agent(s) throughout the elastomeric ring. Matrix rings may also be manufactured using advanced 3D printing or additive manufacturing processes. In some embodiments, this design provides for an exponential or first order release decay, characterized by an initial high release of agent, followed by a lower release rate of agent. More generally, this matrix design provides for drug release that is characterized by a relatively high initial burst release followed by declining drug release rates with time, although other types of kinetics are observed under particular circumstances.

As used herein, the term “reservoir ring,” “exposed-core ring” or “exposed-core type ring” refers to an IVR comprising a reservoir, at least one full or partial length core, surrounded by a sheath. The core and sheath can be made of the same or different materials. For example, the core and sheath can be made of the same or different elastomers. Various active agents can be incorporated into and released from either the sheath or the exposed core. In some embodiments, various active agents can be incorporated into and released from the sheath only, the core only, or both. This design can modulate the drug release rate compared to a matrix ring, often providing for a substantially constant (or zero order) release of agent.

As used herein, the term “elastomer” refers to a polymer network formed when a polymer or a mixture of polymers undergo cross-linking. Commonly, elastomers are formed by chemical cross-linking, and the covalent cross-linking ensures that the elastomer returns to its original configuration when the stress is removed. However, elastomers also extend to certain thermoplastic polymers which exhibit physical cross-links. Irrespective of the type of elastomer, the polymer is typically comprised of monomeric units, which are linked together to form the polymer network. The monomeric units can comprise carbon, hydrogen, oxygen, silicon, halogen, or a combination thereof. Also, a elastomer is typically a material exhibiting viscoelasticity (i.e. both viscosity and elasticity) and with weak intermolecular forces, generally low Young's modulus (E), and high failure strain compared with other materials.

As used herein, the term “prevent,” “prevention,” “treat,” “treatment” or “therapy” as used herein refers to the intravaginal administration of an active agent or a combination thereof, for example, through IVR, in order to prevent or reduce the likelihood of being pregnancy or acquiring STIs, and/or inhibiting or destroying the growth of a microbial organism including that increase the risk of STIs and other adverse reproductive outcomes.

As used herein, the term “active,” “pharmacologically active,” “therapeutically active,” “pharmaceutically active” or “physiologically active” to describe “agent” as used herein means any chemical material or compound used alone or in combination suitable for intravaginal administration, for example, through IVR, which induces a desired effect (e.g., systemic effect).

As used herein, the term “effective,” “pharmacologically effective,” “therapeutically effective,” “pharmaceutically effective” or “physiologically effective” amount of an active agent as used herein means a non-toxic but sufficient amount of a chemical material or compound used alone or in combination with one or more other materials or compounds, when intravaginally administrated, for example, through IVR, to provide the desired therapeutic effect.

As used herein, the activity of a “contraceptive,” or its effectiveness in preventing pregnancy may be determined by sperm motility inhibition or reduction by the agents released from the IVR after it is intravaginally administered to a female subject.

As used herein, the term “synergy,” “synergism,” “synergistic effect” or “synergistic action” as used herein means an effect of the interaction of the actions of two or more active agents such that the result of the combined action is greater than expected as a simple additive combination of the two or more agents acting separately.

As understood by one of skill in the art, the effective amount of an agent, or the amount percentage or concentration of such agent used in the IVR, can vary depending upon a variety of factors, including for example, the actual contraceptive/antimicrobial activity of each agent in the IVR towards the target to be treated, the synergistic effect of the combination of two or more such agents, the type of the IVR, and the manner of delivery of such agent (e.g., whether the dosage form is intended for extended release). Such activity can be determined according to conventional methods and the amounts of active ingredients formulated accordingly.

The term “pharmaceutically acceptable excipient” or “excipient” used herein refers to carriers or vehicles, buffers, gel forming agents or thickening agents, suspending agents, emollients, moisturizers, solubilizers, stabilizers, pH adjusters (also referred as pH modulating agents), release enhancers and preservatives, which do not cause significant irritation to an organism and do not abrogate the biological activity and properties of the applied active agent.

The term “carriers” or “vehicles” used herein refers to carrier materials suitable for intravaginal administration through IVRs and includes any such material known in the art, e.g., any liquid, gel, solvent, liquid diluent, solubilizer or the like, which is non-toxic and does not interact with other components of the composition in a deleterious manner. Examples of suitable carriers include water, alcohols, mineral oil, silicone, liquid sugars, waxes, petroleum jelly, and a variety of other oils and polymeric materials.

The term “release enhancers” used herein refers to compounds that enhance the rate of release of the agent(s) from the IVR. Such compounds include, but are not limited to, polyvinylpyrrollidone (PVP or povidone), modified cellulose ethers (e.g., hydroxyethylcellulose, hydroxypropylcellulose and hydroxypropylmethylcellulose), microcrystalline cellulose, polyacrylic acid, carbomer, alginic acid, carrageenan, cyclodextrins, dextrin, guar gum, gelatin, xanthan gum and sugars (e.g., monosaccharides such as glucose, fructose and galactose, and disaccharides such as lactose, maltose and fructose).

As used herein, the term “release” or “release rate” refers to the amount or concentration of the active agent which leaves the IVR in any defined time period. “Sustained release” or “sustained release rate” refers to release sufficient to provide contraceptive properties and/or antimicrobial properties over a specific (e.g., an extended) time period.

In one aspect, the present disclosure is directed to an IVR for intravaginally administering a therapeutically effective amount of a non-hormonal active agent or a combination thereof to a female subject.

The female subject may be a female mammal including a female human being.

In one embodiment, IVR may be a matrix type and comprise a therapeutically effective amount of at least one non-hormonal agent dispersed throughout an elastomer.

In another embodiment, the IVR may be an exposed-core type, and comprise a therapeutically effective amount of at least one non-hormonal agent dispersed in at least one core elastomer, and/or sheath elastomer.

Examples of elastomers include, but are not limited to, silicones (organo polysiloxanes), polyethylene vinyl acetate copolymer (EVA), styrene-butadiene-styrene block copolymers, polyphosphazenes, poly(isoprene), poly(isobutylene), polybutadienes, polyurethanes, nitrile rubbers, neoprene rubbers and mixtures thereof. Silicone elastomers, also known as silicone rubbers, are particularly preferred.

As used herein, the term “silicone” or “polysiloxane” refers to any of various compounds containing alternate silicon and oxygen atoms in either a linear or cyclic arrangement usually with one or two organic groups attached to each silicon atom. For example, polysiloxanes include substituted polysiloxanes, and diorganopolysiloxanes such as diarylpolysiloxanes and dialkylpolysiloxanes such as dimethylpolysiloxane.

In one embodiment, the elastomer is present in a concentration of about 50% to about 99% by total weight of the IVR. In one embodiment, the elastomer is present in a concentration of about 70% to about 99%, preferably about 90% to about 99% by total weight of the IVR. In one embodiment, the elastomer is present in a concentration of about 95% by total weight of the ring, or about 97% by total weight of the ring.

In one embodiment, the IVR may be a matrix type, and may comprise silicone elastomer (SE) in a concentration of about 50% to about 99%, preferably about 65% to about 90%, and more preferably, about 70% to about 80% by total weight of the IVR. Nonhormonal agent is distributed in the SE prior to final mixing.

In one embodiment, the IVR may be an exposed-core type, and may comprise thermoplastics such as polyethylene vinyl acetate containing 40% EVA (EVA40) or thermoplastic polyurethane (TPU) in a concentration of about 50% to about 99%, preferably about 55% to about 90%, and more preferably, about 55% to about 80% by total weight of the IVR, mixed with the nonhormonal agent. Thermoplastic polymers are ground using cryogenic grinding methods to produce a powdered polymer from stock granulate. Nonhormonal agent powders are weighed in appropriate ratios with the EVA40 or TPU powder and mixed using an appropriate method.

Examples of a non-hormonal agent include, but are not limited to, a copper component, a zinc component and a lactide component. The copper component may be metallic copper, copper oxide, a copper salt or a copper ion-ligand complex. Metallic copper may be in the form of copper microparticles or copper nanoparticles. Examples of copper salts include, but are not limited to, anhydrous copper sulfate and a copper sulfate hydrate (such as CuSO₄·5H₂O). The copper component preferably may be anhydrous copper sulfate. The zinc component may be metallic zinc, zinc oxide or a zinc salt. Metallic zinc may be in the form of zinc microparticles or zinc nanoparticles. Examples of zinc salts include, but are not limited to, anhydrous and hydrated forms, such as zinc acetate, zinc acetate hydrate (including Zn(OAc)₂·2H₂O), zinc formate, zinc formate hydrate, zinc lactate, zinc lactate hydrate (including zinc lactate dihydrate), zinc chloride, zinc chloride hydrate, anhydrous zinc sulfate, zinc sulfate hydrate (such as ZnSO₄·H₂O), zinc iodide, zinc iodide hydrate, zinc citrate, zinc citrate hydrate, zinc orotate and zinc orotate hydrate. The zinc component preferably may be anhydrous zinc lactate, anhydrous zinc acetate, anhydrous zinc sulfate, or a zinc sulfate hydrate. The zinc component most preferably may be anhydrous zinc lactate. The lactide component may be a lactone, such as lactide, lactic acid, or an oligomeric or polymeric form of lactic acid (lactoyllactic acid or polylactic acid). The lactide component may be in the form of D-lactide (such as (R,R)-D-lactide or (S,S)-D-lactide), (R,S)-meso-lactide, DL-lactide, or a mixture thereof. The lactide component preferably may be DL-lactide.

As used herein, unless specifically defined otherwise, copper sulfate can also be referred to as copper sulfate (anhydrous), CS, or CSA; zinc acetate can also be referred to as zinc acetate (anhydrous), ZnA, or ZA; zinc lactate can also be referred to as zinc lactate (anhydrous), ZnL, ZL, or ZLA; lactide can also be referred to as DL-lactide, LT or L; and lactic acid can also be referred to LA.

Milling may be performed on the non-hormonal agent before being used as a material in the IVR manufacturing. In one embodiment, zinc acetate and lactide may require milling before being used as materials for the IVR manufacture. Generally, the unmilled non-hormonal agent may have a particle size distribution wherein 90% have a particle size of greater than 100 μm and less than 1,000 μm, while the milled non-hormonal agent may have a particle size distribution wherein 90% have a particle size of less than 50 μm, preferably less than 30 μm, more preferably less than 20 μm, such as 10 μm. In some embodiments, the unmilled non-hormonal agent may have a particle size distribution wherein greater than 90% of the particles fall within the size range 100 to 1,000 μm, while the milled non-hormonal agent may have a particle size distribution wherein greater than 90% of the particles have size less than 100 μm, preferably less than 50 μm, more preferably less than 20 μm, such as 10 μm. The particle size of the non-hormonal agent may be varied to alter the release rate characteristics of IVR.

In one embodiment, the IVR may comprise at least two non-hormonal agents each in a therapeutically effective amount and dispersed throughout an elastomer. The non-hormonal agents are selected from a copper component, a zinc component, or a lactide component. In one embodiment, the IVR may comprise a lactide component and at least one of a copper component or a zinc component each in a therapeutically effective amount and dispersed throughout an elastomer. In one embodiment, the IVR may comprise a copper component, a zinc component and a lactide component dispersed throughout an elastomer each in a therapeutically effective amount.

In one embodiment, about 100 mg to about 1,600 mg, and preferably about 250 mg to about 1,600 mg, of a copper component is present in the IVR. In one embodiment, about 100 mg to about 1,600 mg, and preferably about 250 mg to about 1,600 mg, of a zinc component is present in the IVR. In one embodiment, about 100 mg to about 1,600 mg, and preferably about 250 mg to about 1,600 mg, of a lactide component is present in the IVR. In one embodiment, about 100 mg to about 1,600 mg, and preferably about 250 mg to about 1,600 mg, of a lactide component, and in combination with at least 100 mg to about 1,600 mg, and preferably about 250 mg to about 1,600 mg, of a copper component, or 100 mg to about 1,600 mg, and preferably about 250 mg to about 1,600 mg, of a zinc component are present in the IVR. In one embodiment, about 100 mg to about 1,600 mg, and preferably about 250 mg to about 1,600 mg, of a lactide component, 100 mg to about 1,600 mg, and preferably about 260 mg to about 1,600 mg, of a copper component, and 100 mg to about 1,600 mg, and preferably about 250 mg to about 1,600 mg, of a zinc component are present in the IVR.

In one embodiment, about 5 wt % to about 50 wt %, about 5 wt % to about 45 wt %, about 5 wt % to about 40 wt %, or about 5 wt % to about 35 wt %, and preferably about 5 wt % to about 30 wt %, or about 10 wt % to about 20 wt %, of a copper component is present in the IVR. In one embodiment, about 5 wt % to about 50 wt %, about 5 wt % to about 45 wt %, about 5 wt % to about 40 wt %, or about 5 wt % to about 35 wt %, and preferably about 5 wt % to about 30 wt %, or about 10 wt % to about 20 wt %, of a zinc component, is present in the IVR. In one embodiment, about 5 wt % to about 50 wt %, about 5 wt % to about 45 wt %, about 5 wt % to about 40 wt %, or about 5 wt % to about 35 wt %, and preferably about 5 wt % to about 30 wt %, or about 10 wt % to about 20 wt %, of a lactide component is present in the IVR. In one embodiment, about 5 wt % to about 50 wt %, about 5 wt % to about 45 wt %, about 5 wt % to about 40 wt %, or about 5 wt % to about 35 wt %, and preferably about 5 wt % to about 30 wt %, or about 10 wt % to about 20 wt %, of a lactide component, and in combination with at least 5 wt % to about 50 wt %, about 5 wt % to about 45 wt %, about 5 wt % to about 40 wt %, or about 5 wt % to about 35 wt %, and preferably about 5 wt % to about 30 wt %, or about 10 wt % to about 20 wt %, of a copper component, or about 5 wt % to about 50 wt %, about 5 wt % to about 45 wt %, about 5 wt % to about 40 wt %, or about 5 wt % to about 35 wt %, and preferably about 5 wt % to about 30 wt %, or about 10 wt % to about 20 wt %, of a zinc component are present in the IVR. In one embodiment, about 5 wt % to about 50 wt %, about 5 wt % to about 45 wt %, about 5 wt % to about 40 wt %, or about 5 wt % to about 35 wt %, and preferably about 5 wt % to about 30 wt %, or about 10 wt % to about 20 wt %, of a lactide component, about 5 wt % to about 50 wt %, about 5 wt % to about 45 wt %, about 5 wt % to about 40 wt %, or about 5 wt % to about 35 wt %, and preferably about 5 wt % to about 30 wt %, or about 10 wt % to about 20 wt %, of a copper component, and about 5 wt % to about 50 wt %, about 5 wt % to about 45 wt %, about 5 wt % to about 40 wt %, or about 5 wt % to about 35 wt %, and preferably about 5 wt % to about 30 wt %, or about 10 wt % to about 20 wt %, of a zinc component are present in the IVR.

The IVR may comprise other pharmaceutically compatible agents in a suitable amount. Such agents include pharmacologically active agents, as well as pharmacologically inactive agents known in the art as pharmaceutically acceptable excipients.

Examples of pharmacologically active agents include, but are not limited to, any suitable antimicrobial agents including antibacterial, antifungal, antiprotozal and antiviral agents known in the art.

Suitable antibacterial agents include, but at not limited to, Acrosoxacin, Amifloxacin, Amoxycillin, Ampicillin, Aspoxicillin, Azidocillin, Azithromycin, Aztreonam, Balofloxacin, Benzylpenicillin, Biapenem, Brodimoprim, Cefaclor, Cefadroxil, Cefatrizine, Cefcapene, Cefdinir, Cefetamet, Cefmetazole, Cefprozil, Cefroxadine, Ceftibuten, Cefuroxime, Cephalexin, Cephalonium, Cephaloridine, Cephamandole, Cephazolin, Cephradine, Chlorquinaldol, Chlortetracycline, Ciclacillin, Cinoxacin, Ciprofloxacin, Clarithromycin, Clavulanic Acid, Clindamycin, Clofazimine, Cloxacillin, Danofloxacin, Dapsone, Demeclocycline, Dicloxacillin, Difloxacin, Doxycycline, Enoxacin, Enrofloxacin, Erythromycin, Fleroxacin, Flomoxef, Flucloxacillin, Flumequine, Fosfomycin, Isoniazid, Levofloxacin, Mandelic Acid, Mecillinam, Metronidazole, Minocycline, Mupirocin, Nadifloxacin, Nalidixic Acid, Nifuirtoinol, Nitrofurantoin, Nitroxoline, Norfloxacin, Ofloxacin, Oxytetracycline, Panipenem, Pefloxacin, Phenoxymethylpenicillin, Pipemidic Acid, Piromidic Acid, Pivampicillin, Pivmecillinam, Prulifloxacin, Rufloxacin, Sparfloxacin, Sulbactam, Sulfabenzamide, Sulfacytine, Sulfametopyrazine, Sulphacetamide, Sulphadiazine, Sulphadimidine, Sulphamethizole, Sulphamethoxazole, Sulphanilamide, Sulphasomidine, Sulphathiazole, Temafloxacin, Tetracycline, Tetroxoprim, Tinidazole, Tosufloxacin, Trimethoprim and salts or esters thereof.

Suitable antifungal agents include, but are not limited to, Bifonazole, Butoconazole, Chlordantoin, Chlorphenesin, Ciclopirox Olamine, Clotrimazole, Eberconazole, Econazole, Fluconazole, Flutrimazole, Isoconazole, Itraconazole, Ketoconazole, Miconazole, Nifuroxime, Tioconazole, Terconazole, Undecenoic Acid and salts or esters thereof.

Suitable antiprotozoal agents include, but are not limited to, Acetarsol, Azanidazole, Chloroquine, Metronidazole, Nifuratel, Nimorazole, Omidazole, Propenidazole, Secnidazole, Sinefingin, Tenonitrozole, Temidazole, Tinidazole and salts or esters thereof.

Suitable antiviral agents include, but are not limited to, Acyclovir, Brivudine, Cidofovir, Curcumin, Dapivirine, Desciclovir, 1-Docosanol, Edoxudine, Fameyclovir, Fiacitabine, Ibacitabine, Imiquimod, Lamivudine, Penciclovir, Valacyclovir, Valganciclovir and salts or esters thereof.

When employed, the other active agents described above may be present in an amount of about 0.5 to about 40 w/w % and preferably about 2.5 to about 15 w/w % of the IVR.

Examples of pharmaceutically acceptable excipients include, but are not limited to, carriers or vehicles, buffers, gel forming agents or thickening agents, suspending agents, emollients, moisturizers, solubilizers, stabilizers, pH adjusters (also referred as pH modulating agents, release enhancers and preservatives, which do not cause significant irritation to an organism and do not abrogate the biological activity and properties of the applied active agent.

Suitable release enhancers include, but are not limited to, polyvinylpyrrollidone (PVP or povidone), modified cellulose ethers (e.g., hydroxyethylcellulose, hydroxypropylcellulose and hydroxypropylmethylcellulose), microcrystalline cellulose, polyacrylic acid, carbomer, alginic acid, carrageenan, cyclodextrins, dextrin, guar gum, gelatin, xanthan gum and sugars (e.g., monosaccharides such as glucose, fructose and galactose, and dissaccharides such as lactose, maltose and fructose). When employed, the release enhancer may be present in an amount of about 0.5 to about 40 w/w % and preferably about 2.5 to about 15 w/w % of the IVR.

The IVR may have a weight of about 2 g to about 15 g, preferably about 5 g to about 10 g, and more preferably about 8 g.

The IVR may have any shape and be of any dimensions compatible for intravaginal administration to a female subject. Such a ring can be self-inserted into the vagina, where it is held in place due to its shape and inherent elasticity. In one embodiment, the manufactured IVR has an outer diameter of 50 to 60 mm. In another embodiment, the IVR has an outer diameter of about 50 mm, about 51 mm, about 52 mm, about 53 mm, about 54 mm, about 55 mm, about 56 mm, about 57 mm, about 58 mm, about 59 mm or about 60 mm. In another embodiment, the IVR has a cross-sectional diameter of 4.0 to 10 mm. In yet another embodiment, the IVR has a cross-sectional diameter of about 4.0 mm, about 4.5 mm, about 5.0 mm, about 5.5 mm, about 5.6 mm, about 6.0 mm, about 6.2 mm, about 6.5 mm, about 7.0 mm, about 7.1 mm, about 7.2 mm, about 7.3 mm, about 7.4 mm, about 7.5 mm, about 7.6 mm, about 7.7 mm, about 7.8 mm, about 7.9 mm, about 8.0 mm, about 8.5 mm, about 9.0 mm, or about 9.5 mm.

The IVR may swell in aqueous media, depending on the type and loading of the actives.

The IVR may be analyzed for in vitro release by using various release media such as water, buffers or simulated vaginal fluid. For example, the qualification of copper and zinc ions released from the IVR may be analyzed by atomic absorption spectroscopy (AAS) or inductively coupled plasma atomic emission spectroscopy (ICP-OES), and the qualification of lactide and its hydrolysis product lactic acid released from the IVR may be analyzed by high performance liquid chromatography (HPLC) with spectroscopic detection. In preferred embodiments, copper, zinc, lactic acid or lactide are determined when the IVR is placed into deionized water (60 rpm, 37° C.) over 30 days.

The IVR of the present disclosure provides long-term controlled release of copper or its ion, zinc or its ion, or lactic acid. Surprisingly, an IVR comprising a combination of at least two of a copper component, a zinc component, or a lactide component provides an increased release of copper or its ion, zinc or its ion, or lactic acid, as compared to that comprising a single component. Particularly, release amount or release rate may be greater when the IVR comprises a non-hormonal agent in a salt form, compared to that comprising a non-hormonal agent in a metallic nanoparticle form, presumably due to the increased water solubility of the salt.

The IVR of the present disclosure, when in use, is capable of releasing lactic acid (or the lactide equivalent) into the vaginal space, at an initial release rate of 1-30 mg, preferably 1-25 mg, most preferably 1-20 mg, as determined in vitro, over the initial 24 hr period followed by a “maintenance” release rate of 0.15-15 mg, preferably 0.20-10 mg, most preferably 0.20-8 mg, as determined in vitro, on a daily basis for at least the following 28-day period. In vitro pH studies have also demonstrated that the lactic acid released from the IVR results in a reduction in pH.

The IVR of the present disclosure, when in use, is capable of releasing copper or its ion, or zinc or its ion, into the vaginal space, at an initial release rate of 1-50 mg, preferably 1-30 mg, most preferably 1-25 mg, as determined in vitro, over the initial 24 hr period followed by a “maintenance” release rate of 0.15-20 mg, preferably 0.20-15 mg, most preferably 0.20-10 mg, as determined in vitro, on a daily basis for at least the following thirty-day period, a “booster” release rate of 1-40 mg, preferably 1-20 mg, most preferably 1-10 mg, as determined in vitro, on a daily basis for at least the following ten-day period, and by another “maintenance” release rate of 0.15-20 mg, preferably 0.20-15 mg, most preferably 0.20-10 mg, as determined in vitro, on a daily basis for at least the following eight-day period.

In one embodiment, optionally in combination with any of the features or embodiments described herein, the IVR of the present disclosure, when in use, is capable of having a sustained and continuous release of lactic acid (or the lactide equivalent) into the vaginal space, at a rate of at least 15 mg/day, preferably at least 20 mg/day, at least 25 mg/day, at least 30 mg/day, at least 35 mg/day, or at least 40 mg/day as determined in vitro, over the first 15 days of a 30 day period. In one embodiment, optionally in combination with any of the features or embodiments described herein, the IVR of the present disclosure, when in use, is capable of having a sustained and continuous release of lactic acid (or the lactide equivalent) into the vaginal space, at a rate of 15 to 110 mg/day, preferably 20 to 105 mg/day as determined in vitro, over the first 15 days.

In one embodiment, the IVR of the present disclosure, when in use, is capable of releasing lactic acid (or the lactide equivalent) into the vaginal space, at a release rate of 5-150 mg/day, preferably 10 to 110 mg/day, more preferably 12-105 mg/day, or 20-150 mg/day as determined in vitro, over a period of 30 days. In one embodiment, the IVR of the present disclosure, when in use, is capable of releasing copper ion into the vaginal space, at a release rate of 1-50 mg/day, preferably 2-35 mg/day, or 5-50 mg/day as determined in vitro, over a period of 30 days. In one embodiment, the IVR of the present disclosure, when in use, is capable of releasing zinc ion into the vaginal space, at a release rate of 1-40 mg/day, preferably 1-20 mg/day, or 2-40 mg/day as determined in vitro, over a period of 30 days.

In one embodiment, the IVR of the present disclosure, comprising 10CSA-10ZLA-10L (a combo of 10 wt % copper sulfate (anhydrous), 10 wt % zinc lactate (anhydrous), and 10 wt % lactide), when in use, is capable of releasing into the vaginal space, lactic acid (or the lactide equivalent) at a release rate of 12-60 mg/day, releasing copper ion at a release rate of 2-20 mg/day, and releasing zinc ion at a release rate of 1-10 mg/day as determined in vitro, over a period of 30 days. In one embodiment, the IVR of the present disclosure, comprising 10CSA-10ZLA-20L (a combo of 10 wt % copper sulfate (anhydrous), 10 wt % zinc lactate (anhydrous), and 20 wt % lactide), when in use, is capable of releasing into the vaginal space, lactic acid (or the lactide equivalent) at a release rate of 12-105 mg/day, releasing copper ion at a release rate of 2-35 mg/day, and releasing zinc ion at a release rate of 1-20 mg/day as determined in vitro, over a period of 30 days. In one embodiment, the IVR of the present disclosure, comprising 5CSA-10ZLA-10L (a combo of 5 wt % copper sulfate (anhydrous), 10 wt % zinc lactate (anhydrous), and 10 wt % lactide), when in use, is capable of releasing into the vaginal space, lactic acid (or the lactide equivalent) at a release rate of 5-50 mg/day, releasing copper ion at a release rate of 1-20 mg/day, and releasing zinc ion at a release rate of 1-8 mg/day as determined in vitro, over a period of 30 days. In one embodiment, the IVR of the present disclosure, comprising 5CSA-20ZLA-20L (a combo of 5 wt % copper sulfate (anhydrous), 20 wt % zinc lactate (anhydrous), and 20 wt % lactide), when in use, is capable of releasing into the vaginal space, lactic acid (or the lactide equivalent) at a release rate of 15-100 mg/day, releasing copper ion at a release rate of 2-20 mg/day, and releasing zinc ion at a release rate of 2-40 mg/day as determined in vitro, over a period of 30 days. In one embodiment, the IVR of the present disclosure, comprising 10CSA-10ZLA-30L (a combo of 10 wt % copper sulfate (anhydrous), 10 wt % zinc lactate (anhydrous), and 30 wt % lactide), when in use, is capable of releasing into the vaginal space, lactic acid (or the lactide equivalent) at a release rate of 20-150 mg/day, releasing copper ion at a release rate of 5-50 mg/day, and releasing zinc ion at a release rate of 2-30 mg/day as determined in vitro, over a period of 30 days. Herein, wt % refers a weight percentage based on a total weight of the IVR.

In one embodiment, the IVR of the present disclosure, when in use, is capable of releasing lactic acid (or the lactide equivalent) into the vaginal space, at an initial release rate of 20-60 mg, preferably 22-55 mg, most preferably 50-55 mg, as determined in vitro, over the first week period followed by a release rate of 30-105 mg, preferably 50-90 mg, most preferably 85-90 mg, as determined in vitro, on a daily basis for the following 7-day period, a release rate of 20-30 mg, preferably 22-30 mg, most preferably 25-30 mg, and a release rate of 12-25 mg, as determined in vitro, on a daily basis for the next following 7-day period.

In one embodiment, the IVR of the present disclosure, when in use, is capable of releasing copper ion into the vaginal space, at an initial release rate of 2-31 mg, preferably 4-31 mg, as determined in vitro, over the first week period followed by a release rate of 2-20 mg, preferably 15-20 mg, as determined in vitro, on a daily basis for the following 7-day period, a release rate of 2-10 mg, preferably 4-10 mg, and a release rate of 2-5 mg, as determined in vitro, on a daily basis for the next following 7-day period.

In one embodiment, the IVR of the present disclosure, when in use, is capable of releasing zinc ion into the vaginal space, at an initial release rate of 1-17 mg, preferably 12-17 mg, as determined in vitro, over the first week period followed by a release rate of 5-9 mg, preferably 7-9 mg, as determined in vitro, on a daily basis for the following 7-day period, a release rate of 2-5 mg, and a release rate of 1-3 mg, as determined in vitro, on a daily basis for the next following 7-day period.

In one embodiment, the release rate of the copper ion from a multi-component IVR of the present disclosure (e.g. a ring comprising a copper component and at least one of a zinc component or a lactide component) is increased as compared to the release rate of copper ion from an IVR comprising a copper component without either a zinc component or a lactide component. For example, in one embodiment, the release rate of copper ion from an IVR of the present disclosure comprising a copper component and either or both of a zinc component or a lactide component is increased at least about 1.5- to about 2-fold per day as compared to the release rate of copper ion from an IVR comprising a copper component without either a zinc component or a lactide component. In another embodiment, the release rate of copper ion from an IVR of the present disclosure comprising a copper component and either a zinc component or a lactide component is increased at least about 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45 or 50-fold per day as compared to the release rate of copper ion from an IVR comprising a copper component without either a zinc component or a lactide component.

In one embodiment, the cumulative release rate of copper ion from an IVR of the present disclosure comprising a copper component and either a zinc component or a lactide component is increased at least about 1.5- to about 2-fold after 7 days, after 14 days, after 21 days, after 30 days, after 60 days, or after 90 days as compared to the cumulative release rate of copper ion from an IVR comprising a copper component without either a zinc component or a lactide component after 7 days, after 14 days, after 21 days, after 30 days, after 60 days, or after 90 days. In one embodiment, the cumulative release rate of copper ion from an IVR of the present disclosure comprising a copper component and either a zinc component or a lactide component is increased at least about after 7 days, after 14 days, after 21 days, after 30 days, after 60 days, or after 90 days as compared to the cumulative release rate of copper ion from an IVR comprising a copper component without either a zinc component or a lactide component after 7 days, after 14 days, after 21 days, after 30 days, after 60 days, or after 90 days. In another embodiment, the cumulative release rate of copper ion from an IVR of the present disclosure comprising a copper component and either a zinc component or a lactide component is increased at least about 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45 or 50-fold after 7 days, after 14 days, after 21 days, after 30 days, after 60 days, or after 90 days as compared to the cumulative release rate of copper ion from an IVR comprising a copper component without either a zinc component or a lactide component after 7 days, after 14 days, after 21 days, after 30 days, after 60 days, or after 90 days. In one embodiment, the cumulative release rate of copper ion from an IVR of the present disclosure comprising a zinc component or a lactide component is increased by about 2-fold to 50-fold, by about 2-fold to about 25-fold, by about 2-fold to about 20-fold, by about 2-fold to about 15-fold, by about 2-fold to about 10-fold, by about 2-fold to about 5-fold, by about 5-fold to about 25-fold, by about 5-fold to about 20-fold, by about 5-fold to about 15-fold, by about 5-fold to about 10-fold, by about 10-fold to about 25-fold, or by about 10-fold to about 20-fold after 7 days, after 14 days, after 21 days, after 30 days, after 60 days, or after 90 days as compared to the cumulative release rate of copper ion from an IVR comprising a copper component without either a zinc component or a lactide component after 7 days, after 14 days, after 21 days, after 30 days, after 60 days, or after 90 days.

In one embodiment, the release rate of the zinc ion from a multi-component IVR of the present disclosure (e.g. a ring comprising a zinc component and at least one of a copper component or a lactide component) is increased as compared to the release rate of zinc ion from an IVR comprising a zinc component without either a copper component or a lactide component. For example, in one embodiment, the release rate of zinc ion from an IVR of the present disclosure comprising a zinc component and either or both of a copper component or a lactide component is increased at least about 1.5- to about 2-fold per day as compared to the release rate of zinc ion from an IVR comprising a zinc component without either a copper component or a lactide component. In another embodiment, the release rate of zinc ion from an IVR of the present disclosure comprising a zinc component and either a copper component or a lactide component is increased at least about 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45 or 50-fold per day as compared to the release rate of zinc ion from an IVR comprising a zinc component without either a copper component or a lactide component.

In one embodiment, the cumulative release rate of zinc ion from an IVR of the present disclosure comprising a zinc component and either a copper component or a lactide component is increased at least about 1.5- to about 2-fold after 7 days, after 14 days, after 21 days, after 30 days, after 60 days, or after 90 days as compared to the cumulative release rate of zinc ion from an IVR comprising a zinc component without either a copper component or a lactide component after 7 days, after 14 days, after 21 days, after 30 days, after 60 days, or after 90 days. In one embodiment, the cumulative release rate of zinc ion from an IVR of the present disclosure comprising a zinc component and either or both of a copper component or a lactide component is increased at least about 10-fold after 7 days, after 14 days, after 21 days, after 30 days, after 60 days, or after 90 days as compared to the cumulative release rate of zinc ion from an IVR comprising a zinc component without either a copper component or a lactide component after 7 days, after 14 days, after 21 days, after 30 days, after 60 days, or after 90 days. In another embodiment, the cumulative release rate of copper ion from an IVR of the present disclosure comprising a zinc component and either a copper component or a lactide component is increased at least about 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45 or 50-fold after 7 days, after 14 days, after 21 days, after 30 days, after 60 days, or after 90 days as compared to the cumulative release rate of zinc ion from an IVR comprising a zinc component without either a copper component or a lactide component after 7 days, after 14 days, after 21 days, after 30 days, after 60 days, or after 90 days. In one embodiment, the cumulative release rate of zinc ion from an IVR of the present disclosure comprising a copper component or a lactide component is increased by about 2-fold to 50-fold, by about 2-fold to about 25-fold, by about 2-fold to about 20-fold, by about 2-fold to about 15-fold, by about 2-fold to about 10-fold, by about 2-fold to about 5-fold, by about 5-fold to about 25-fold, by about 5-fold to about 20-fold, by about 5-fold to about 15-fold, by about 5-fold to about 10-fold, by about 10-fold to about 25-fold, or by about 10-fold to about 20-fold after 7 days, after 14 days, after 21 days, after 30 days, after 60 days, or after 90 days as compared to the cumulative release rate of zinc ion from an IVR comprising a zinc component without either a copper component or a lactide component after 7 days, after 14 days, after 21 days, after 30 days, after 60 days, or after 90 days.

In one embodiment, release rates are stable following 3 months of storage. In one embodiment, release rates are stable following 6 months of storage. In one embodiment, release rates are stable following 12 months of storage. In one embodiment, release rates are stable following 24 months of storage. In one embodiment, release rates are stable following 36 months of storage.

In one aspect, the present disclosure is directed to a method of making an IVR for intravaginally administering an active ingredient or a combination thereof to a female subject.

The matrix-type ring may comprise a non-hormonal agent or a combination thereof in silicone elastomer. Matrix-type rings may alternatively comprise a polyurethane or EVA polymer, or a mixture thereof.

Silicone matrix-type rings may be manufactured by mixing the non-hormonal agents, optionally with other active agents and excipients, into the silicone material in different medical grade along with a suitable crosslinking agent (such as propylorthosilicate (NPOS)) and a catalyst (such as platinum) if any, injecting the mixture into suitably designed injection molds (such as Babyplast® injection molding machine fitted with custom ring mold assemblies), and optionally applying heat to cause the silicone mix to cure/crosslink forming an elastomer. More generally, silicone matrix-type rings may be manufactured by mixing the non-hormonal agents, optionally with other active agents and excipients, into a suitably formulated silicone elastomer system (which may include base silicone polymers, crosslinking agents and cure catalysts, and others suitable for medical use) and then injecting the mixture into an injection molding machine.

Thermoplastic matrix-type rings may be prepared by: (i) compounding the non-hormonal agents, optionally with other active agents and excipients, into a thermoplastic polymer (such as ethylene vinyl acetate (EVA) copolymer or a polyurethane) using a twin-screw compounder, (ii) pelletizing the mixture, and (iii) injection molding to form IVRs using an injection molding machine fitted with custom injection molds. In one embodiment, cooling of the compounded material may be performed using air and/or chilled haul off belts. The use of cooling water bath may not be possible due to the water-soluble nature of the non-hormonal active ingredients. Once cooled, the rod output from the extruder may be cut into small pellets and used in downstream injection molding operations. In another embodiment, thermoplastic matrix-type rings may be manufactured by extrusion, cutting the extrudate to length, and welding the ends to form a ring-shaped device, similar to the commercial manufacturing method for the contraceptive IVR NuvaRing®.

More complex core-exposed rings may be manufactured by using different drug-loaded core segments (either polymer with embedded active or copper tubing) and a polymeric membrane having holes/windows partially exposing the drug-loaded core segments.

In one embodiment, a non-hormonal agent or a combination thereof may be placed in the core with a blank sheath. In some embodiments, one agent is placed in the core and the other agents are placed in the sheath. In one embodiment, the lactide component may be placed in the core and the copper component and the zinc component may be placed in the sheath. In other embodiments, the copper component or the zinc component may be placed in the sheath. In other embodiments, two components may be placed in the core and one component in the sheath.

A two-step process may be used to prepare the core-exposed ring. In one embodiment, a core ring is first produced in the same manner as the matrix ring described above. This core is then placed into a second mold cavity designed to hold the core in the center of the cavity and additionally produce openings (windows) in the sheath layer as it is being formed.

Curing temperatures and times vary, depending on the particular elastomer(s) used. Curing temperatures are preferably below the melting point of the non-hormonal agents used. For example, the curing temperature may vary between room temperature (15-25° C.) and about 150° C., preferably within the range of about 60-125° C., and more preferably within the range of about 90-115° C. The curing time may vary between a few seconds and several hours, depending on such factors as the elastomer(s) used and the curing temperature.

Rings may be characterized by microscopy, differential scanning calorimetry (DSC), thermal gravimetric analysis (TGA), atomic absorption spectroscopy (AES), ICP-OES, and HPLC to probe the nature and behavior of the actives in the devices, to assess release, and to assess drug-drug and drug-polymer interactions. Mechanical testing of IVRs may be conducted according to established methods, such as using a Shimadzu EZ Test Universal Tester fitted with custom test jigs. For example, different mechanical tests may be optimized for the IVR geometries and used to mechanically characterize IVR performance: Shore M hardness, 28-day static compression, 5-20 mm compression, 1000-cycle compression and elongation at break.

In one aspect, the present disclosure is directed to a method of preventing unintended pregnancy, and preventing and treating a disease caused by bacterial, fungal, and viral infection by applying an IVR for intravaginally administering a pharmaceutical effective amount of an active agent or a combination thereof to a female subject.

In one embodiment, the disease may be caused by a sexually transmitted infection.

The IVR is described above. In one embodiment, the IVR of the present disclosure may be useful in inhibiting sperm motility, and/or preventing or inhibiting an infection caused by human immunodeficiency virus (HIV) such as HIV-1, herpes simplex virus (HSV) such as HSV-2, N. gonorrhoeae, G. vaginalis (associated with bacterial vaginosis (BV), chlamydia, etc.

In one embodiment, the therapeutic effects, including disease inhibition and contraceptive effect, are achieved when at least two of copper ion, zinc ion or lactic acid are released from the IVR in use into the vaginal space.

In one embodiment, the therapeutic effect is achieved when copper ion and zinc ion are released from the IVR in use into the vaginal space, as determined in vitro, at a molar ratio of 5:1 to 1:5, preferably 1:1 to 1:3, and more preferably 1:2 in a vaginal fluid each day for 24 days after an initial first day period of use. In one embodiment, the therapeutic effect is achieved when copper ion and lactide acid are released from the IVR in use into the vaginal space, as determined in vitro, at a molar ratio of 5:1 to 1:5, preferably 1:1 to 1:3, and more preferably 1:2 in a vaginal fluid each day for 24 days after an initial first day period of use. In one embodiment, the therapeutic effect is achieved when zinc ion and lactide acid are released from the IVR in use into the vaginal space, as determined in vitro, at a molar ratio of 5:1 to 1:5, preferably 2:1 to 1:2, and more preferably 1:1 in a vaginal fluid each day for 24 days after an initial first day period of use. In one embodiment, the therapeutic effect is achieved when copper ion, zinc ion and lactic acid is released from the IVR in use, as determined in vitro, in a molar ratio of 1:0.5:2 to 1:4:8, preferably 1:0.8:3, 1:1:2, 1:1:3, 1:1.25:5, 1:1.5:5, 1:3:7, and more preferably 1:1.5:5.25, in a vaginal fluid each day for 24 days after an initial first day period of use.

In one embodiment, optionally in combination with any of the features or embodiments described herein, preferably when the IVR comprises a combination of CSA-ZLA-L, the therapeutic effect is achieved when copper ion and zinc ion are released from the IVR in use into the vaginal space, as determined in vitro, at a molar ratio of 1:1 to 1:6, preferably 1:3 to 1:6, and more preferably 1:3.5 to 1:5.5 in a vaginal fluid each day for 24 days after an initial first day period of use. In one embodiment, optionally in combination with any of the features or embodiments described herein, preferably when the IVR comprises a combination of CSA-ZLA-L, the therapeutic effect is achieved when copper ion and lactic acid are released from the IVR in use into the vaginal space, as determined in vitro, at a molar ratio of 1:1 to 1:6, preferably 1:3 to 1:6, and more preferably 1:1.66 to 1:5.25 in a vaginal fluid each day for 24 days after an initial first day period of use. In one embodiment, optionally in combination with any of the features or embodiments described herein, preferably when the IVR comprises a combination of CSA-ZLA-L, the therapeutic effect is achieved when zinc ion and lactide acid are released from the IVR in use into the vaginal space, as determined in vitro, at a molar ratio of 3:1 to 1:1, preferably 2.5:1 to 1.1:1, and more preferably 2.2:1 to 1.05:1 in a vaginal fluid each day for 24 days after an initial first day period of use. In one embodiment, optionally in combination with any of the features or embodiments described herein, preferably when the IVR comprises a combination of CSA-ZLA-L, the therapeutic effect is achieved when copper ion, zinc ion and lactic acid is released from the IVR in use, as determined in vitro, in a molar ratio of 1:1:1 to 1:6:6, preferably 1:3:3 to 1:6:6, and more preferably 1:3.5:1.66 to 1:5.5:5.25, even more preferably to 1:5.5:5.25, in a vaginal fluid each day for 24 days after an initial first day period of use.

In one embodiment, optionally in combination with any of the features or embodiments described herein, preferably when the IVR comprises a combination of CSA-ZLA-L, the copper ion and zinc ion are comprised in the IVR at a molar ratio of 1:1 to 1:6, preferably 1:3 to 1:6, and more preferably 1:3.5 to 1:5. In one embodiment, optionally in combination with any of the features or embodiments described herein, preferably when the IVR comprises a combination of CSA-ZLA-L, the copper ion and lactic acid arec comprised in the IVR at a molar ratio of 1:1 to 1:6, preferably 1:3 to 1:6, and more preferably 1:1.66 to 1:5.25. In one embodiment, optionally in combination with any of the features or embodiments described herein, preferably when the IVR comprises a combination of CSA-ZLA-L, the zinc ion and lactic acid are comprised in the IVR at a molar ratio of 3:1 to 1:1, preferably 2.5:1 to 1.1:1, and more preferably 2.2:1 to 1.05:1 in a vaginal fluid each day for 24 days after an initial first day period of use. In one embodiment, optionally in combination with any of the features or embodiments described herein, preferably when the IVR comprises a combination of CSA-ZLA-L, the copper ion, zinc ion and lactic acid are comprised in the IVR at a molar ratio of 1:1:1 to 1:6:6, preferably 1:3:3 to 1:6:6, and more preferably 1:3.5:1.66 to 1:5.5:5.25, even more preferably to 1:5.5:5.25.

In one embodiment, the IVR of the present disclosure may be useful in maintaining the vaginal health.

The IVR of the present disclosure may be administered into the vagina of a female subject about 1 to about 6 weeks prior to sexual intercourse. In one embodiment, the IVR may be administered into the vagina of a female subject about 1 to about 15 days prior to sexual intercourse.

### Examples

### Methods for Preparing IVRs

IVRs of the present disclosure may be manufactured by any method known by those skilled-in-the-art, but preferably using injection molding or extrusion technologies, and more preferably by reaction injection molding of silicone elastomer systems. The term ‘injection molding’ refers to manufacturing processes for producing parts/devices from either thermoplastic or thermosetting materials using suitably designed injection molds. Examples of thermoplastic materials include polyethylene, polyethylene vinyl acetate (PEVA) and certain polyurethanes; examples of thermosetting materials include silicone rubbers/elastomers. Without limitation, matrix-type silicone elastomer rings containing the active agents may be prepared by (i) adding and mixing the active agents into one or more components of the silicone system (e.g., base, crosslinking agent, catalyst, excipient, dispersant, etc.) (ii) injecting the mix into suitably designed injection molds, and (iii) optionally, applying heat to cause the silicone mix to cure/crosslink forming an elastomer.

These methods generally comprise dispersing the active agents and an elastomer, e.g., polysiloxane, in an appropriate solvent or dispersing agent, e.g., silicone liquid, and curing the rings with a platinum catalyst, e.g., a platinum-siloxane complex, thereby preparing a platinum-catalyzed ring. Any of the well-known elastomers, e.g., polysiloxanes, described supra, may be used to prepare the IVRs of the present disclosure. In one embodiment, an elastomer, e.g., polysiloxane, for use in the methods of the present disclosure, is an addition cure dimethylsiloxane silicone elastomer, comprising mixtures of hydride-vinyl-terminated polydimethylsiloxane molecules. In another embodiment, an elastomer, e.g., polysiloxane, for use in the methods of the present disclosure, is a condensation cure silicone elastomer, comprising hydroxy-terminated dimethylpolysiloxane molecules and a tetraalkoxylsilane crosslinking agent. In another embodiment, the elastomer, e.g., polydimethylsiloxane, for use in the methods of the present disclosure, is MED-8470. In certain embodiments, the methods further comprise use of a cross-linker, e.g., hydride functional polydimethylsiloxane or dimethylmethylhydrogen polysiloxane cross-link. In another embodiment, the silicone elastomer may comprise organic groups other than methyl groups, e.g., fluorine, phenyl, etc.

In one embodiment, the method further comprises catalyzing the rings in a ring mold. The mold can then be opened, following which the IVR is removed and trimmed. Ring molds, are preferably coated with, for example, Teflon™ or an electrolytically applied metalised coating. Ring molds may be constructed of hardened carbon steel, stainless steel, aluminum, or any other material deemed to be appropriate. It is appreciated that the mold dimensions and designs impart the physical shape of the IVR, for example, a partial or complete ring, or any other desired shape. Preferably, the device has a partial or complete toroidal shape, more preferably a partial or complete torus shape, or a substantially cylindrical shape. By toroid is meant a ring-like body generated by rotating any closed loop (including an ellipse, a circle or any irregular curve) about a fixed line external to that loop. The toroid shape may be a complete or partial toroid. By torus is meant a ring-like body generated by rotating a circle about a fixed line external to the circle. The torus shape may be a complete or partial ring-like shape. The geometric characteristics of the mold and IVRs can be varied as required by the use.

Alternatively, the IVR device, or components thereof, may be prepared by extrusion processes, e.g., co-extrusion or blend extrusion, well known to those skilled in the art (see, e.g., U.S. Pat. No. 5,059,363, the entire contents of which are incorporated herein by reference). In preferred embodiments, the IVR devices may be made by a matrix-type LSR4350 silicone elastomer manufactured at 115° C. for 3 mins used in the examples described herein below.

### In Vitro Studies of Sperm Motility Reduction for CS-ZA-LA

Active Pharmaceutical Ingredient (API): CS—CuSO₄, ZA-Zn(OAC)₂, LA-lactic acid

### Sperm Motility Assay

Fresh human sperm samples were obtained the day of the experiment. 100 mM stock solutions of each API in saline were prepared in advance. Immediately prior to the experiment, solutions of each API concentration to be tested were prepared via dilution with saline. These include solutions of multiple APIs added together.

The effect of each API on sperm motility and viability was determined using human donors, adding 200 μL of API's diluted in sterile saline to 50 μL of fresh semen. For motility assessments, the incubation mixture was incubated for 5 minutes and then 100 sperm were counted under the microscope by two andrology-trained researchers and classified as either non-motile or motile. If motile, the sperm were further classified according to the nature of their motility—from barely twitching with no forward progression to progressively motile. Each experiment was repeated at least three times on different days using different donors to account for natural variations in sperm properties.

### Data Analysis

The % motility for each experimental condition was calculated by normalizing the fraction of motile sperm in the experimental groups to the fraction of motile sperm in the saline control treated group, setting 100% to the average fraction of motile sperm from control experiments. For example, the % motility at concentration c would be:

$$\% {Motility}_{c} = \frac{\# Motile {Sperm}_{c}}{\# Motile {Sperm}_{saline}}$$

Once % motility data was obtained, the synergy was measured with Compusyn software. See Chou et al., CompuSyn for drug combinations: PC software and user's guide: a computer program for quantitation of synergism and antagonism in drug combinations, and the determination of IC₅₀. ED₅₀ and LD₅₀ values, which is incorporated herein by reference in its entirety. Motility data for individual API concentration as well as all double and triple API solutions was entered into the program and analyzed according to the Chou-Talalay Median Effect Principle. Table 1 shows calculated EC values.

TABLE 1
EC₅₀ and Ec₉₀ Values for APIs

| Compound | EC₅₀ (mM) | EC₉₀ (mM) |
|---|---|---|
| CS | 11.81 | 18.66 |
| ZA | 19.02 | 33.16 |
| LA | 20.54 | 28.68 |

Table 1 shows that CS is the most effective among the APIs followed by ZA, and then LA. However, LA has the smallest % difference between its EC₅₀ and EC₉₀ concentrations, indicating that past a certain threshold its motility inhibiting efficacy begins to rapidly increase.

Further, it indicated that all APIs worked fast to immobilize sperm, and their activity was noted at 30 seconds.

Next, the synergy between each of the APIs was tested to determine if there were any synergistic effects between compounds which would allow for reduced dosages. Each of the three possible two-compound combinations were tested, followed by the combination of all three APIs. The two-compound combinations were tested using serial dilutions of a fixed ratio of APIs corresponding to the approximate ratio of their EC₅₀ values (2:1 for ZA:CS and LA:CS and 1:1 for ZA:LA). Synergy was calculated with Compusyn using the Chou-Talalay Median Effect approach and represented as a CI (Combination Index) value. CI values <1 indicate synergy while CI values >1 indicate antagonism. The smaller the CI value for a combination, the greater the degree of synergy. Table 2 shows the EC values of the two-compound combinations as well as their corresponding CI values at those concentrations. It is important to note that the EC concentrations refer to the sum of the two-component concentrations (e.g., the 13.97 mM EC₅₀ of 1:1 ZA:LA refers to 6.985 mM of LA+6.985 mM of Zn). Table 2 shows the calculated EC and CI values for each 2-API combination.

TABLE 2
EC₅₀, EC₉₀ and CI values for 2-API Combinations

| | | EC₅₀ | | | |
|---|---|---|---|---|---|
| Ratio | Combo | (mM) | EC₉₀ (mM) | CI at EC₅₀ | CI at EC₉₀ |
| 2:1 | ZA:CS | 11.89 | 20.73 | 0.78 | 0.80 |
| 2:1 | LA:CS | 10.50 | 17.98 | 0.64 | 0.55 |
| 1:1 | LA:ZA | 13.97 | 24.36 | 0.73 | 0.42 |

Table 2 shows that all three combinations exhibited significant synergy, reducing the concentrations of each individual API needed to reach the same effect level. LA also appears to be the most significant API at increasing synergy, producing significantly lower CI values in its two combinations than the ZA:CS combination. Table 2 also illustrates that the CI values of the LA combinations increase with increasing API concentration and effect level.

After the two-API combinations were complete, the full three-way synergy was tested using a 3×3×3 checkerboard style assay to test all 27 permutations of three chosen concentrations for each of the three APIs. Based on the results of the previous 2-compound assays and practical considerations of API ring release rates, it was decided to use higher concentrations of lactic acid than CS and ZA in hopes of maximizing lactic acid's effect on synergy and decreasing the required release levels of CS and ZA. For CS and ZA, the three concentrations chosen were equivalent to ¼, ⅓ and ½ of the respective API's EC₅₀ value. For LA, these fractions were doubled, resulting in concentrations ½, ⅔ 1× the EC₅₀ value. The actual concentrations used are listed in Table 3.

TABLE 3
Concentrations Used in 3-API Synergy Checkboard Assays

| | [CS] (mM) | [ZA] (mM) | [LA] (mM) |
|---|---|---|---|
| Dose 1 | 2.95 | 4.75 | 10.26 |
| Dose 2 | 3.94 | 6.34 | 13.70 |
| Dose 3 | 5.90 | 9.51 | 20.54 |

From the resulting data, the estimated effects of each of the individual API were isolated by averaging % reduction in motility and the CI values for each of the API concentrations tested. With this 3×3×3 checkerboard assay, each single concentration was tested against nine permutations of the other two APIs, meaning that each cell in Table 4 represents the average of the nine combinations involving the specified API concentration.

TABLE 4
Isolated Effects of Individual APIs
from 3-API Combination Assays

| | CS | ZA | LA | | | |
|---|---|---|---|---|---|---|
| | Average | Average | Average | CS | ZA | LA |
| | Motility | Motility | Motility | Average | Average | Average |
| | Reduction | Reduction | Reduction | CI | CI | CI |
| Dose 1 | 55.71% | 63.47% | 54.75% | 0.59 | 0.48 | 0.69 |
| Dose 2 | 74.95% | 73.77% | 70.74% | 0.44 | 0.47 | 0.51 |
| Dose 3 | 86.68% | 80.09% | 91.84% | 0.37 | 0.45 | 0.22 |

Table 4 shows that out of the three APIs, CS appears to be the most effective and synergistic at relatively low concentrations, showing a steady increase in effect and CI across a very narrow concentration range. ZA shows a similar, albeit less pronounced ability to reduce sperm motility, but shows almost no dose-response effect on the CI value. LA shows similar effects as CS and ZA at doses 1 and 2 but sees a significant jump in motility reduction and especially CI at the highest concentration—its EC₅₀ was 20.54 mM. Altogether, this data indicates that CS and ZA are much more effective at inducing synergy and reducing sperm motility at lower percentages of their EC₅₀ values, while LA requires higher relative concentrations to achieve the same effects. However, at these high lactic acid concentrations, it becomes incredibly potent at increasing synergy between all three APIs.

The three-API combinations that produced a motility reduction of >90% were selected and used in conjunction with the previous insights into the nature of the synergy to select a final combination of concentrations to move forward with. Table 5 shows all these combinations.

TABLE 5
3-API Combinations that Caused >90% Motility Reduction

| [CS] | [ZA] | [LA] | Motility | |
|---|---|---|---|---|
| (mM) | (mM) | (mM) | Reduction | CI |
| 3 | 10 | 21 | 93.35 | 0.26 |
| 4 | 5 | 21 | 93.55 | 0.21 |
| 4 | 6 | 21 | 99.91 | 0.12 |
| 4 | 10 | 21 | 100 | 0.14 |
| 6 | 5 | 21 | 100 | 0.14 |
| 6 | 6 | 14 | 92.82 | 0.32 |
| 6 | 6 | 21 | 100 | 0.15 |

Based on the data, it was determined that a target intravaginal concentration of 4 mM CS, 6 mM ZA, and 21 mM LA would be the ideal mixture to aim for in further ring formulations. The 4 mM of CS shows significant abilities to inhibit sperm motility while being present at less than half of its original EC₅₀, greatly reducing its potential toxicity and reducing the required ring-release rate. ZA was shown to be the least important API in terms of reducing motility and inducing synergy, so the low level of 6 mM ZA was chosen to minimize the challenges of ring-release while still allowing enough ZA to act synergistically with the other APIs to keep their concentrations minimal. Finally, LA was selected at the relatively high level of 21 mM as it showed a remarkable increase in effect and synergy at high concentrations, showed the least amount of toxicity, and proved much more capable of being released at high rates from rings in preliminary studies.

### Studies of Different Zinc Salts

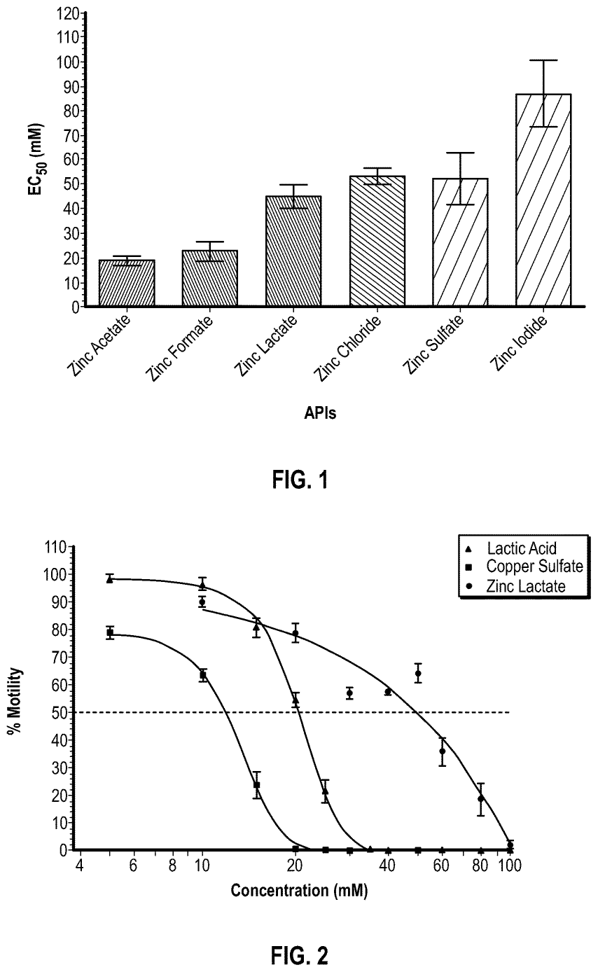

Several zinc salts were tested for sperm motility reduction by using the sperm motility assay described above, and the activities were shown in FIG. 1. It was noted that zinc acetate when released from the rings resulted in unpleasant smell, and zinc formate had solubility issues. Zinc lactate (ZL) was selected based on its overall profile on the sperm motility reduction, odor, solubility and pH.

### In Vitro Studies of Sperm Motility Reduction and Sperm-Mucus Penetration for CS-ZL-LA

API: CS—CuSO₄, ZL-zinc lactate, LA-lactic acid

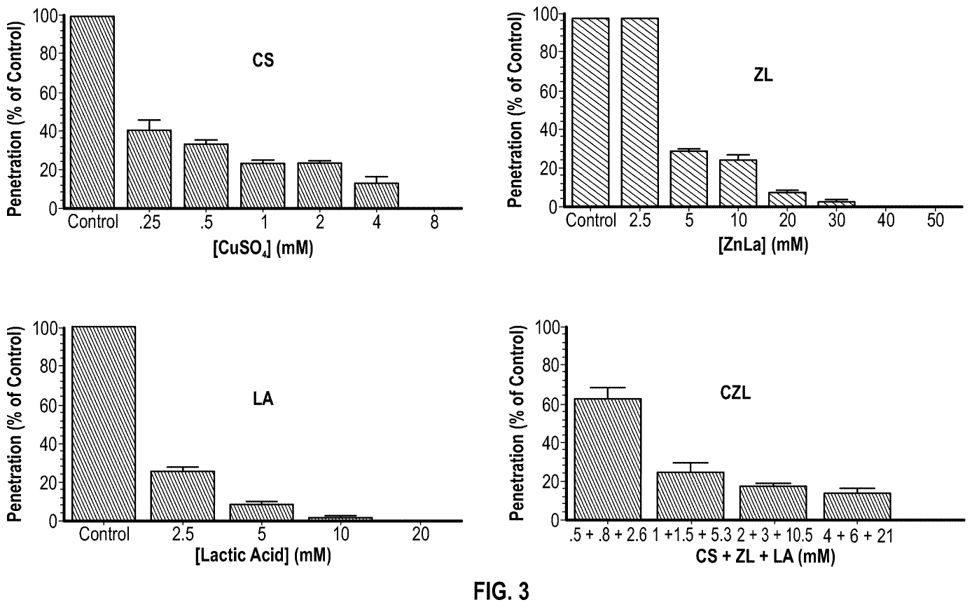

The effect of each API on sperm motility and viability was determined according to the method described above. FIG. 2 shows the results of each API of CS, ZL and LA for sperm motility testing, which all inhibit sperm motility at mM concentrations. Table 6 shows calculated EC values for each API of CS, ZL and LA.

TABLE 6
EC50 and Ec90 Values for APIs

| Compound | EC₅₀ (mM) | EC₉₀ (mM) |
|---|---|---|
| CS | 12 | 19 |
| ZL | 45 | 94 |
| LA | 21 | 29 |

After Single API dose response curves and EC₅₀ values were determined, synergy of two-API and three-API combinations were investigated using checkerboard assays testing all permutations of 3-4 concentrations of each API. Synergy was calculated utilizing the Chou-Talalay median effect principle and Compusyn software which quantifies synergy as a combination index (CI) for each combination. A CI<1 signifies synergy, a CI>1 signifies antagonism and a CI=1 signifies a purely additive effect.

Sperm-Cervical Mucus Penetration Test (SMPT) Experiments (Ivic et al., Critical evaluation of methylcellulose as an alternative medium in sperm migration tests. Human Reproduction, Volume 17, Issue 1, January 2002, Pages 143-149) were performed by placing semen+API solutions in a capillary tube filled with 1% methylcellulose (the validated surrogate, methyl cellulose, currently used in diagnostic laboratories) and measuring the migration of the sperm after 30 minutes. SMPT test using fresh human ejaculates (sperm) after treatment with test API or vehicle only treated (control) is used to determine the extent to which treated sperm migrates through the cervical mucus or some surrogate medium (such as methylcellulose which allows for a high degree of precision and accuracy, as well as reproducibility because the concentration and characteristics do not vary, unlike cervical mucus from different women or different times of the cycle) filled in the glass capillary tubes compared to untreated sperm.

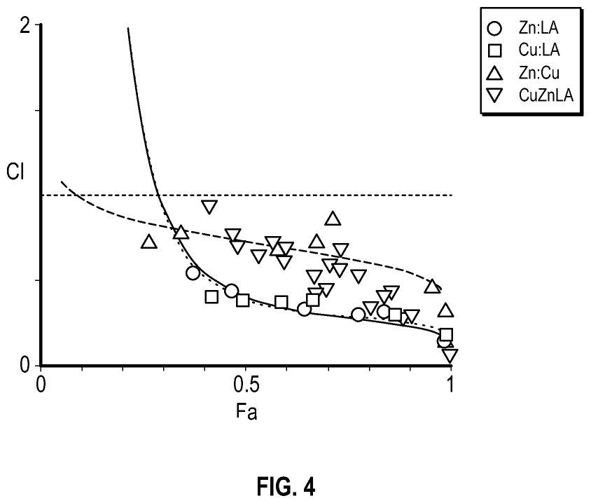

FIG. 3 shows the results of SMPT results for single API and the CS-ZL-LA combination. The calculated data of sperm motility reduction and sperm-mucus penetration inhibition were shown in Table 7. Lower API concentrations were able to inhibit sperm penetration through cervical mucus (methylcellulose) compared to the concentrations required to inhibit sperm motility.

TABLE 7
Sperm Motility and Sperm-Mucus Penetration

| | Reduction of | | | | |
|---|---|---|---|---|---|
| | Sperm Motility | | | Inhibition of | |
| | EC90 [95% | | | Sperm Mucus Penetration | |
| | CI***] - mM | | *MIC - mM | **EC90 [95% CI***] - mM | |
| | (concentration to | | (concentration to | (Migration of vanguard sperm | |
| | inhibit total sperm | | inhibit progressive | in methyl cellulose spiked | |
| API | motility) | | sperm motility) | with API) | |
| CS | 19 | [17 to 20] | 10 | 5 | [3 to 8] |
| ZL | 94 | [81 to 110] | 30 | 13 | [9 to 18] |
| LA | 29 | [27 to 30] | 20 | 5 | [3 to 7] |

| | | | |
|---|---|---|---|
| CS + ZL + LA* | 4 + 22 + 21 | 3 + 22 + 10 | 0.15 + 5 + 1.5 |

*Minimum Inhibitory Concentration (MIC) is defined as the concentration required to inhibit progressive sperm motility.
**For triple API combos, EC90 could not be calculated empirically, and instead the smallest tested combination achieving 90% inhibition was selected.
***CI: confidence interval

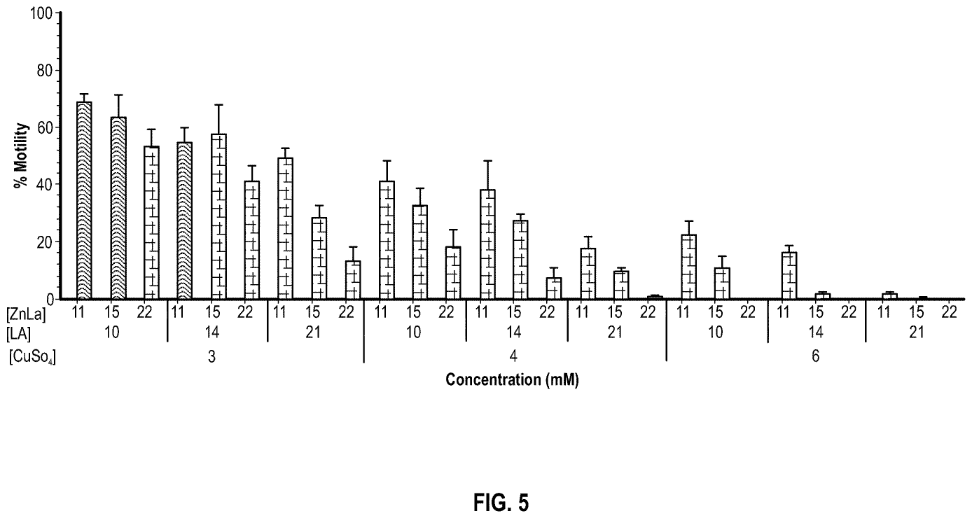

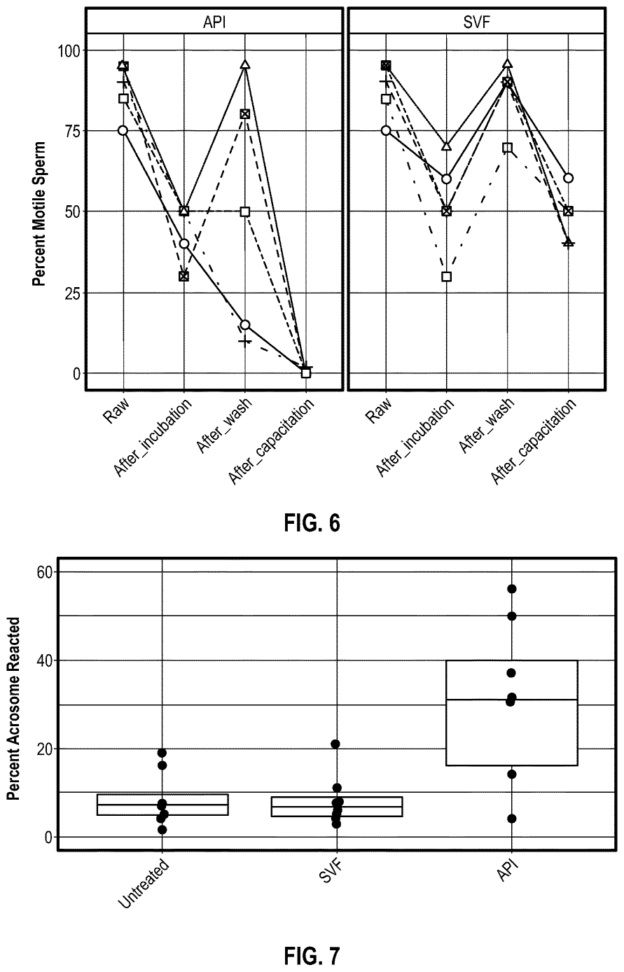

All tested CS/ZL/LA combinations showed some degree of synergy on sperm motility reduction and sperm mucus penetration inhibition. See FIG. 4, showing synergy plotted against efficacy (stated as the fraction affected; Fa), where Cu refers to CS, and Zn refers to ZL; FIG. 5, showing motility inhibition for CS-ZL-LA combination, where each bar represents a CS-ZL-LA combination with solid bars showing progressively motility, and hashed bars showing no progressive motility based on an average of four experimental repeats; and Table 8, showing calculated motility reduction and CI values, where CI values <1 show synergy, and the smaller the CI, the greater the magnitude of synergy.

TABLE 8
3-API Combinations that Caused >90%
Motility Reduction using CS-ZL-LA:

| | | | Motility | |
|---|---|---|---|---|
| [CS] | [LA] | [ZL] | Reduction | |
| (mM) | (mM) | (mM) | (%) | CI |
| 4 | 14 | 22 | 93 | 0.51 |
| 4 | 21 | 15 | 90 | 0.60 |
| 4 | 21 | 22 | 99 | 0.24 |
| 6 | 10 | 22 | 100 | 0.23 |
| 6 | 14 | 15 | 98 | 0.28 |
| 6 | 14 | 22 | 100 | 0.24 |
| 6 | 21 | 11 | 98 | 0.30 |
| 6 | 21 | 15 | 100 | 0.24 |
| 6 | 6 | 22 | 100 | 0.27 |

As shown from FIG. 4, CS+LA and ZL+LA had the highest levels of synergy, with an average CI<0.2. CS+LA and ZL+LA also showed almost identical CI/Fa curves, which suggests a similar mechanism of action. CS+ZL showed little synergy, especially at low concentrations. CS+ZL+LA showed significant synergy at high Fa levels, but slightly less synergy than CS+LA or ZL+LA at lower concentrations.

As shown from Table 8, there was a significant correlation between increasing LA dose and increasing synergy (p<0.0001), while ZL and CS showed no such correlation. CS-ZL-LA combinations reduced API concentrations needed for 90% motility inhibition by ⅕ for CS and ZL. Ability to inhibit progressive motility started at even lower concentrations for individual API and combos. From FIG. 3 and Table 7, SMPT results showed similar reductions in API concentrations needed in CS-ZL-LA combos and suggested even lower concentrations may be sufficient for contraceptive efficacy than is required to inhibit motility.

### Studies of Sperm Capacitation for CS-ZL-LA

API: CS—CuSO₄, ZL-zinc lactate, LA-lactic acid

Capacitation, the process of sperm maturation within the female reproductive tract, is critical for acrosomal exocytosis and fertilization. Hence, capacitation is a compelling candidate for contraceptive development.

### Sperm Processing and API Treatment

Fresh semen samples were obtained by masturbation from consenting, healthy men 21-40 years of age. Samples were allowed to liquefy at 37° C. for at least 30 minutes. Next, 200 μL of semen was combined with either 50 μL API (CS-ZL-LA combination: 3.2 mM CS, 9.6 mM ZL, 16.8 mM LA), or 50 μL of simulated vaginal fluid (SVF) (vehicle control) (Owen et al., A Vaginal Fluid Simulant. Contraception 1999, 59, 91-95, doi:10.1016/S0010-7824(99)00010-4), and mixed by gentle trituration using a large orifice pipette. An untreated negative control group was also maintained. Samples were incubated for 5 minutes at 37° C.

To remove seminal plasma and API, first sperm was centrifuged through enhance S-Plus cell isolation medium (Vitrolife) at 300 g for 10 minutes, and the supernatant was removed leaving behind a loose pellet of sperm. Sperm pellets were resuspended by gentle trituration in the remaining 100 μL of medium, then transferred to a new tube, resuspended with 4 mL modified human tubal fluid (mHTF, Fujifilm), and centrifuged at 600 g for 10 minutes. The resultant pellet was then resuspended in 150 μL mHTF, and sperm concentration was calculated using a hemocytometer. Depending on the experiment, sperm were subsequently incubated at a concentration of 10 million/mL for 3 hours in either capacitating (5 mM 2-hydroxypropyl-β-cyclodextrin, 2-OHCD) or non-capacitating (no cyclodextrin) conditions. These capacitation conditions have previously been shown to be effective in human sperm (Cardona et al., Localization Patterns of the Ganglioside GM1 in Human Sperm Are Indicative of Male Fertility and Independent of Traditional Semen Measures. Mol Reprod Dev 2017, 84, 423-435, doi:10.1002/mrd.22803).

### Motility Assessment

Sperm motility was assessed in the raw ejaculate, after 5-minute incubation with each of treatments (API, SVF, and untreated control), after the wash step, and after 3-hour incubation in capacitating conditions. To assess motility, 15 μL of sperm was examined at 40× on a 37° C. warmed slide. The number of progressively motile sperm was visually assessed and documented.

### Sperm Membrane Fluidity Assessment

Assessment of changes in membrane fluidity was performed using a modified form of the Cap-Score Test (Androvia Lifesciences, Mountainside, NJ). To test a man's fertility, the Cap-Score Test involved incubation of sperm with 2-OHCD, a mediator of sterol efflux and stimulus for capacitation, and then a percentage of sperm that responded to those conditions was established by quantifying the sperm having different patterns of localization of the membrane ganglioside G_{M1} (Moody et al., Validation of a Laboratory-Developed Test of Human Sperm Capacitation. Mol Reprod Dev 2017, 84, 408-422, doi:10.1002/mrd.22801). The test used Cholera Toxin B (CTB)- and proprietary fixative conditions to induce rearrangement of the G_{M1} into those patterns. In more rigid membranes that have not undergone sterol efflux, CTB:488 binds to G_{M1} and trigger G_{M1} rearrangement to the post-acrosomal plasma membrane and lower equatorial segment of the sperm head. In more fluid membranes signifying response to the 2-OHCD through sterol efflux, CTB:488 labels G_{M}1 over the acrosome and does not trigger G_{M1} rearrangement to the post-acrosomal plasma membrane or lower equatorial region; rather, the G_{M1} remains throughout the plasma membrane over the acrosome or even be concentrated in the apical acrosome.

After 3-hour incubation in either capacitating or non-capacitating conditions, sperm were fixed in mHTF overnight at room temperature. Immediately before imaging, 1 μL of CTB:488 (Invitrogen) was added to sperm. Then, 5 μL of CTB:488-stained sperm were placed on a slide and imaged with a Nikon Eclipse TE2000-U fluorescent microscope. For each experimental treatment, the number of sperm exhibiting CTB:488 staining patterns consistent with fluid or rigid membranes were counted. Then the percentage of sperm with CTB:488 staining patterns indicative of high membrane fluidity were calculated.

### Coomassie Staining for Acrosonal Exocytosis

Coomassie staining is a commonly used method for evaluating acrosomal status in fixed sperm. After 3-hour incubation in capacitating conditions, sperm were fixed for 10 minutes in 2% paraformaldehyde. Sperm were then spun at 500 g for 10 minutes. The supernatant was removed, and sperm were resuspended in 500 μL 100 mM ammonium acetate (pH 9.0), and spun again at 500 g for 10 minutes. The supernatant was removed, and the sperm pellet was resuspended in another 500 μL of 100 mM ammonium acetate (pH 9.0). To mount sperm on slides, a circular region was outlined on a clean glass slide using a PAP pen. 40 μL of sperm was added into the demarcated area and allowed to air-dry on a 37° C. slide warmer. To stain the sperm, 150 μL Coomassie working solution (0.22% Coomassie R-250, 50% methanol, 10% acetic acid) was added to dry sperm for 6 minutes. Excess stain was removed, and slides were washed by dipping in ddH₂O. Washed slides were air-dried on a slide warmer. Immediately before scoring, a drop of aqueous mounting media and coverslip were placed on the slide. The number of sperm with intact or reacted acrosomes was counted at 40× using a Nikon Eclipse E200 light microscope.

### Live/Dead Staining

Sperm viability was evaluated using Sybr-14/Propidium iodide staining (LIVE/DEAD Sperm Viability kit, Invitrogen). In this assay, SYBR-14 labeled live sperm with green fluorescence, while propidium iodide labeled membrane-compromised sperm with red fluorescence. First, SYBR-14 stock was diluted 50× in mHTF. Then, 1.6 μL of diluted SYBR-14 was added to sperm and incubated for 10 minutes at 37° C. Subsequently, sperm were treated with 1.6 μL propidium iodide and incubated for an additional 10 minutes at 37° C. Sperm were then plated on a glass bottom dish (Mattek) smeared with Poly-D-Lysine to immobilize sperm. Images were taken with an Olympus IX3-FP confocal microscope with heated stage. The number of sperm exhibiting live or dead/damaged staining patterns was assessed in ImageJ, and the percentage of sperm with a dead/damaged staining pattern was calculated for each group.

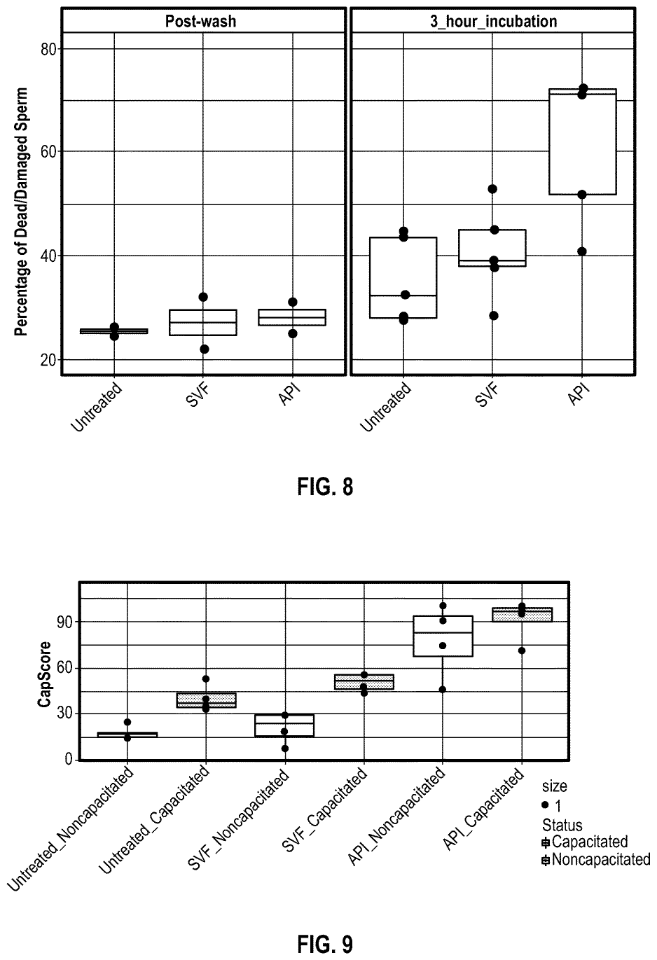

To understand how motility changed throughout the experiment, the percentage of sperm displaying progressive motility at various stages was assessed as: in raw ejaculate, after 5-minute incubation with API (CS-ZL-LA combo: 3.2 mM CS, 9.6 mM ZL, 16.8 mM LA in SVF) or SVF, after washing, and after 3 hours incubation in capacitating conditions. FIG. 6 shows the percentage of motile sperm at different experimental stages for four experiments. API-treated sperm exhibited variable motility recovery after washing. However, even after initial motility recovery, API treated sperm exhibited almost complete cessation of progressive motility after 3-hour incubation in capacitating conditions. This reduction in motility after capacitation was the result of API exposure, as a similar dramatic decrease was not observed in motility in the SVF vehicle controls. API-treated sperm exhibited an almost complete loss of motility after 3-hour incubation in capacitating conditions as demonstrated in FIG. 6. These data showed that even short-term exposure to API has long-term effects on sperm motility.

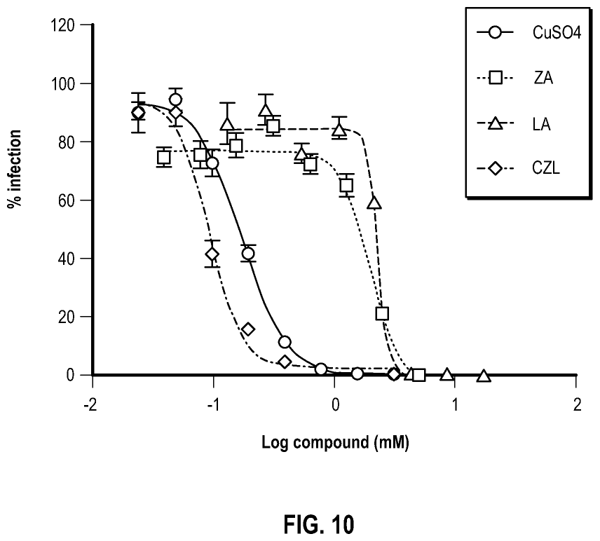

FIG. 7 shows that short-term incubation with API (CS-ZL-LA combo: 3.2 mM CS, 9.6 mM ZL, 16.8 mM LA in SVF) resulted in an increase in spontaneous acrosomal exocytosis after 3-hour incubation in capacitating conditions, compared to those with SVF vehicle control and untreated control.

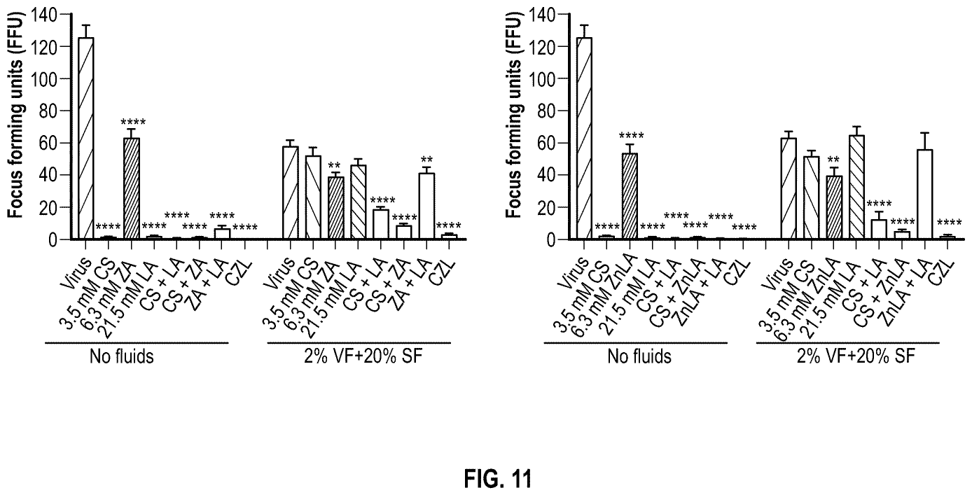

Sperm viability was examined using SYBR-14/propidium iodide staining after the wash step and after 3-hour incubation in capacitating conditions. FIG. 8 showed that API-treated sperm had decreased viability after 3-hour incubation in capacitating conditions compared to those with SVF vehicle control and untreated control. After washing, an effect of API treatment on sperm viability was not observed. However, after 3-hour incubation in capacitating conditions, an increase in the percentage of dead/damaged sperm in the API treated group compared to the untreated and vehicle (SVF) controls was observed. This shows that short-term exposure to APIs had long-term effects on sperm viability.

Cholera Toxin B (CTB) can bind to up to five molecules of the membrane ganglioside G_{M1}. In sperm that have not undergone sterol efflux, and thus have more rigid plasma membranes, binding of CTB to G_{M1} causes G_{M1} rearrangement to the post-acrosomal plasma membrane and lower equatorial segment. In capacitated sperm that have undergone sterol efflux, CTB labels G_{M1} over the acrosome and does not trigger G_{M1} rearrangement. After 5-minute incubation with APIs and 3-hour incubation in capacitating conditions, a very high percentage of sperm exhibited CTB:488 staining patterns associated with increased membrane fluidity. These percentages were well beyond that published for a group of 76 men with proven recent fertility (mean=35.3±7.7%). In fact, this average percentage was higher than any ever recorded in >7000 patient samples at Androvia (Travis, pers. comm.). The vehicle (SVF) and untreated control groups exhibited percentages consistent with what would be expected from a population of fertile men. These results also showed that short-term exposure to APIs had long-term effects on sperm physiology beyond motility.

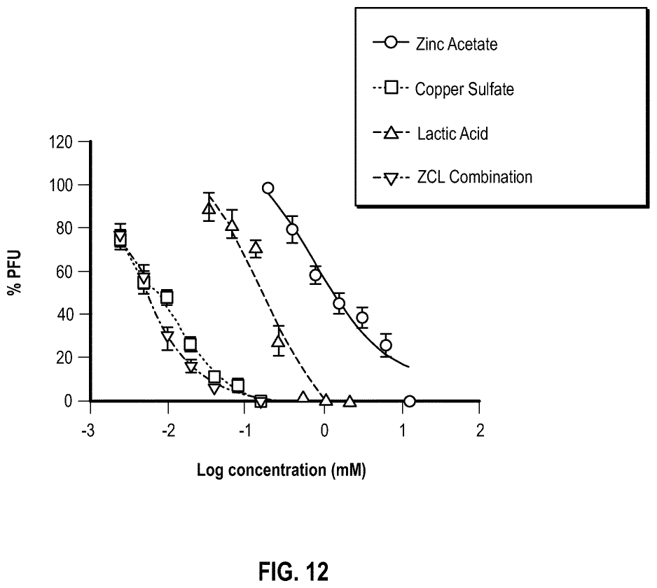

This increase in membrane fluidity was not the result of 2-OHCD mediated sterol efflux or physiologically-relevant capacitation, as API-treated sperm incubated in non-capacitating conditions also showed an extremely high percentage of sperm exhibiting high membrane fluidity. Therefore, API-treated sperm were not physiologically capacitated (having never undergone sterol efflux, which is strictly required for acrosome exocytosis and fertilization), but instead exhibited increased membrane fluidity as the result of an API-induced pathological process. FIG. 9 shows that the increase in membrane fluidity was independent of 2-OHCD induced sterol-efflux. A pathological increase in membrane fluidity may be expected to lead to an increase in spontaneous acrosomal exocytosis, which would also contribute to reduced motility at a population level. The Coomassie staining revealed an increase in the percentage of sperm that had undergone exocytosis in our API-treated group. As seen with the changes in membrane fluidity, this increase in exocytosis reflected a non-physiological process, given that these sperm were not exposed to any relevant triggers for exocytosis.

The present disclosure uncovered new mechanisms of action of the CS-ZL-LA IVR. Short-term exposure to CS-ZL-LA-IVR API (3.2 mM CS, 9.6 mM ZL, and 16.8 mM LA) had multiple long-term effects on sperm fertilizing ability, including decreased motility, decreased viability, increased membrane fluidity, and increased spontaneous acrosome exocytosis. These effects are likely the result of dysregulation of membrane fluidity leading to increased acrosome exocytosis and cell death.

### Studies of Anti-Viral/Anti-Bacteria Activities

Experiments analyzing API interactions in anti-infective assays were done using fixed API ratios.

### In Vitro Anti-HIV Activity Assay

API: CS—CuSO4, ZA-zinc acetate, LA-DL-lactic acid, alone or in combination (CZL which is referred here to CS-ZA-LA)

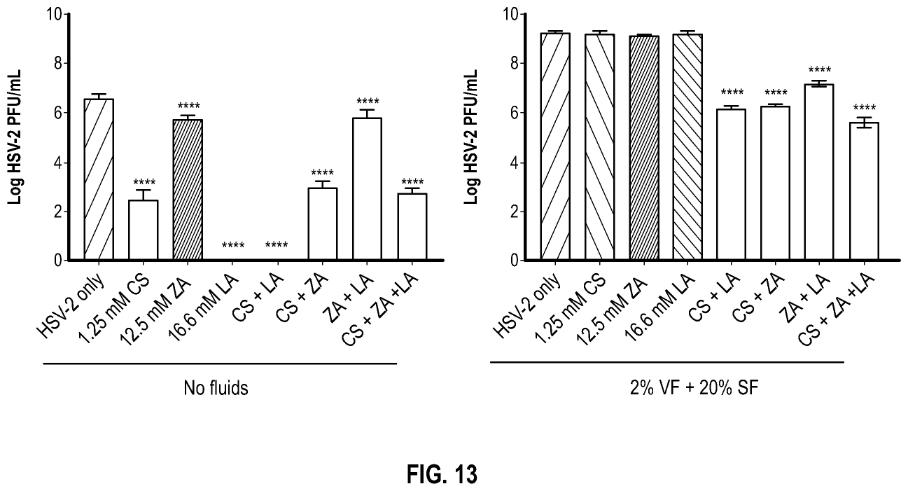

MAGI assay was performed to test anti-HIV-1_{BaL} activity. Different concentrations of each API (CS, ZA, and DL-LA (LA)), alone or in combination, were prepared in saline (0.9% NaCl) and incubated with HIV-1_{BaL} for 30 minutes at 37° C., 5% CO₂ and 98% humidity. Virus controls containing HIV-1_{BaL} in saline were incubated under the same conditions. Following incubation, API-HIV-1_{BaL} mixture was diluted 10-fold and added to TZMbl cells. Following 72-hour incubation, the cells were washed and stained with X-gal and fixed. The virus only control wells had ~200 infected cells. Infected cells were counted using CTL instrument. The APIs demonstrated potent anti-HIV-1_{BaL} activity at sub mM-low mM concentrations, with CSL (CS-ZA-LA) being the most potent (FIG. 10, Table 9).

TABLE 9
Half Maximal Effective Concentrations of APIs.

| | EC50 (95% confidence interval) |
|---|---|
| API | mM |
| CS | 0.183 (0.1548 to 0.2171) |
| ZA | 1.963 (1.628 to 2.735) |
| LA | 2.498 (NA to 2.821) |
| CS-ZA-LA | 0.111 (0.0987 to 0.1253) |
| molar ratio: 1:2:6 | |

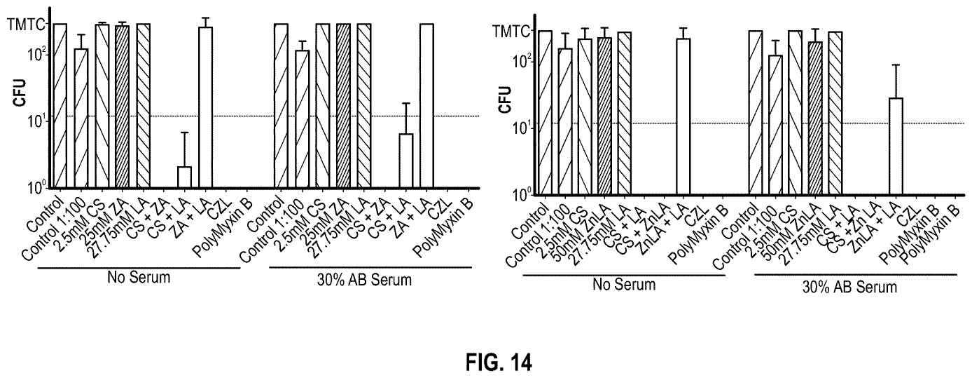

HIV-1_{BaL} was incubated with actives ±2% VF and 20% SF. Then mixtures were serially diluted, applied on TZM-bl cells and viral titer was determined based on FFU. The graph represented 3 experiments (MEAN±SEM) (FIG. 11).

The three actives (alone and combinations) significantly inhibited infection in the absence of the fluids. Activity of individual actives was decreased in the presence of the fluids. However, combinations remained active, with triple combo demonstrating the most potent activity.

### In Vitro Anti-HSV Activity

APIs virucidal activity against HSV-2 Assay.
API: CS—CuSO4, ZA-zinc acetate, LA-lactic acid, alone or in combination (CZL which is referred here to CS-ZA-LA)

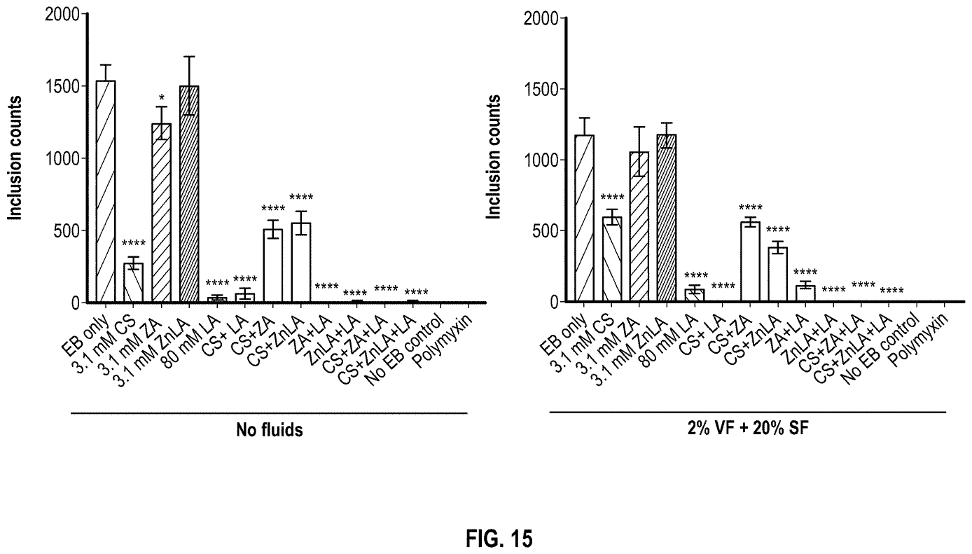

The HSV-2 plaque reduction assay was performed to test the anti-HSV-2 activity of ZA, CS and LA. Different concentrations of each API, alone or in combination, were prepared in saline (0.9% NaCl) and pre-incubated with 10³ HSV-2 G infectious particles for 30 minutes at 37° C., 5% CO₂ and 98% humidity. Virus controls containing HSV-2 in saline were incubated under the same conditions. After incubation all dilutions and controls were diluted 1/10 in DMEM and tittered using the plaque assay. FIG. 12 and Table 10 show the results. FIG. 12 shows the percent of plaque forming units (PFU)±SE at each API concentration versus virus control. As shown in FIG. 12, ZA, CS and LA, alone and in combination, showed a dose-dependent HSV-2 inhibition in the plaque reduction assay. The data represented the average of two independent experiments. In Table 10, the molar ratio of CS:LA:ZA in ZS-CA-LA is 1:20:100. All the APIs alone and in combination had potent virucidal activity against HSV-2 with CS-ZA-LA being the most potent API.

TABLE 10
Half Maximal Effective Concentrations of APIs.

| API | EC₅₀ (95% confidence interval) mM |
|---|---|
| ZA | 0.78 (0.30 to 1.92) |
| CS | 0.009 (0.006 to 0.016) |
| LA | 0.16 (0.10 to 0.26) |
| CS-ZA-LA | 0.002 (0.001 to 0.006) |
| molar ratio: 1:20:100 | |

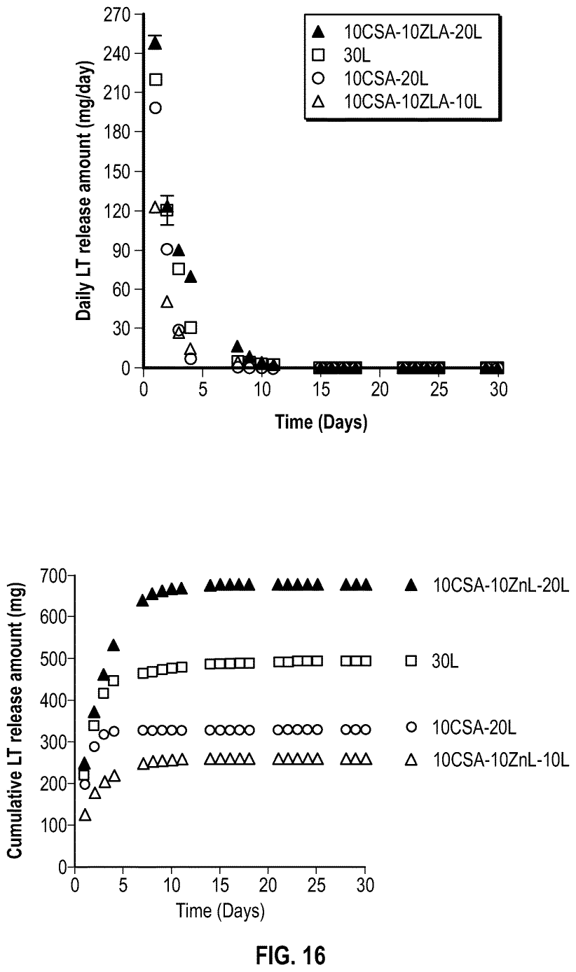

HSV-2 G was incubated with 1.25 mM CS, 12.5 mM ZA, 16.6 mM LA±2% VF and 20% SF for 30 minutes at 37° C., 5% CO₂ and 98% humidity. Then mixtures were serially diluted 1/10, applied on Vero cells and viral titer (PFU/mL) was determined. Control conditions included Vero cells challenged with HSV-2 G in the absence of the actives. FIG. 13 represented three experiments (log of the plaque forming units per milliliter (PFU/mL); MEAN±SEM).

### In Vitro Anti-Vaginalis and Anti-Gonorrhea Activity Assay

API: CS—CuSO4, ZA-zinc acetate, ZL-zinc lactate, LA-lactic acid, alone or in combination (CZL which is referred to CS-ZL-LA)

Minimal cidal concentrations (MCCs) against G. vaginalis and N. gonorrhoeae were determined. Bacteria were resuspended to ~3×10⁸ colony forming units (CFU)/ml (1 McFarland). Serial dilutions of APIs (2× concentrations) were prepared in broth. ~5×10⁵ CFU/ml were exposed to the APIs for 30 minutes at 37° C., 5% CO₂ (tightly capped tube) and then plated undiluted in duplicate or triplicate on agar plates. Plates were cultured aerobically (N. gonorrhoeae) or anaerobically (G. vaginalis). Diluted untreated bacteria were plated to confirm starting inoculum. API concentrations in samples yielding >3 log decrease (99.9%) in colony forming units (CFU) were considered cidal (<500 CFU/ml; <12 colonies on the plate).

For N. gonorrhoeae, CS MCC was 10 mM, ZA MCC was 100 mM, and for LA MCC was 111 mM. For G. vaginalis, CS MCC was 5 mM, ZA MCC was >100 mM, and for LA MCC was 111 mM. No changes in MCCs were observed following testing in the presence of human AB serum.

To determine API interactions, 3-dimentional checkerboard assay was employed using broth microdilution method. Stocks of APIs 4× in broth were prepared and added to 96-well plates containing 10⁵ CFU/well. N. gonorrhoeae was cultured aerobically for 24 hours and G. vaginalis was cultured anaerobically in anaerobic jar for 48 hours. In the end of incubation, bacteria were gently mixed and fixed with PFA. Optical density (OD) at 650 nm was acquired. OD from control wells containing broth only and broth with APIs was subtracted from ODs in the wells containing bacteria and APIs. Fractional inhibitory combination index (FICI) was calculated. FICI=MIC of drug A in combination/MIC of drug A alone+MIC of drug B in combination/MIC of drug B alone+MIC of drug C in combination/MIC of drug C alone. FICI≤0.5—synergy, 0.5<FICI<4—no interaction, ≥4—antagonism. No interaction/weak synergy was noted for both pathogens.

5×10⁵ CFU/mL of N. gonorrhea was incubated with 2.5 mM CS, 25 mM ZA, 50 mM ZL and 27.75 mM LA in single, double, and triple combinations of these actives ±30% AB serum for 30 minutes at 37° C., 5% CO₂ and 98% humidity. The selected concentrations were 3-4 fold lower than the determined MCC for each single API. Untreated bacteria and bacteria exposed to Polymyxin B (64 ug/mL) were positive and negative controls, respectively. After incubation, the bacteria were plated in duplicate on GC agar plates supplemented with Isovitalex (25 μL/plate). The untreated control was diluted 1:100 before plating. After 24-hour incubation at 37° C. with 5% CO₂, colonies were counted using Image J Software. Dilution yielding to more than 3 log reductions (99.9%) in bacteria viability were considered cidal. The figures represented four experiments (Log of CFU count; MEAN±SD).

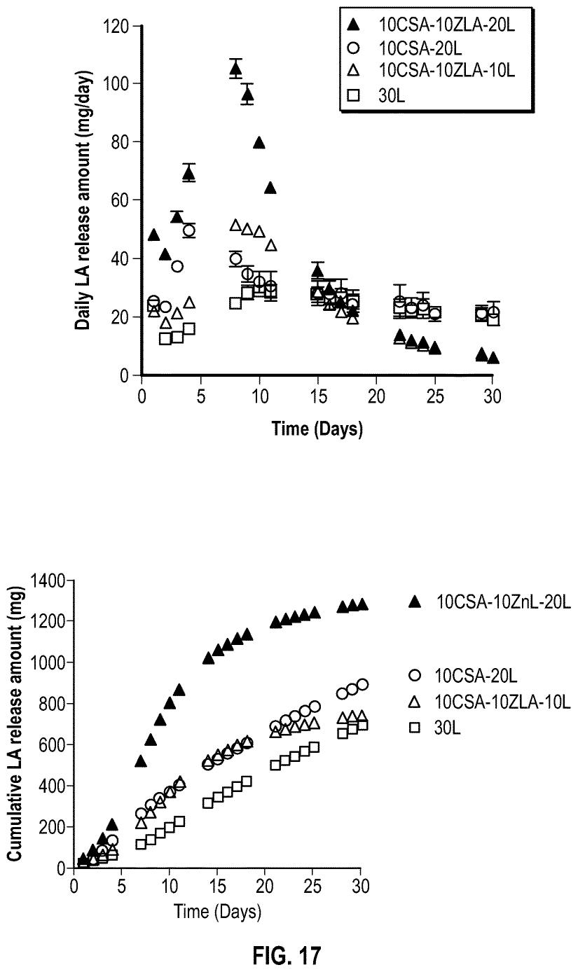

FIG. 14 and Table 11 showed the results for N. gonorrhoeae. As shown in FIG. 14, CS/ZA, CS/LA, and CS-ZA-LA combination at subcidal concentrations resulted in cidal effect. CS 10 mM and DL-LA 111 mM were MCC against N. gonorrhoeae±human serum; however, ZL was not inhibitory. Dual and triple actives combinations at sub-MCC resulted in cidal activity. Table 11 showed the MCC of single actives ±10% and 30% AB serum.

TABLE 11
MCC of single actives ± 10% and 30% AB serum

| | No Serum | 10% AB Serum | 30% AB Serum |
|---|---|---|---|
| MCC | (mM) | (mM) | (mM) |
| CS | 10 | 10 | 10 |
| ZA | 100 | 100 | >100 |
| ZL | >100 | >100 | >100 |
| LA | 111 | 111 | 111 |

In Vitro Bactericidal Activity Against Chlamydial trachomatis (C. trachomatis) Assay
API: CS—CuSO4, ZA-zine acetate, ZL-zinc lactate, LA-DL-lactic acid, alone or in combination (CZL which is referred here to CS-ZL-LA)

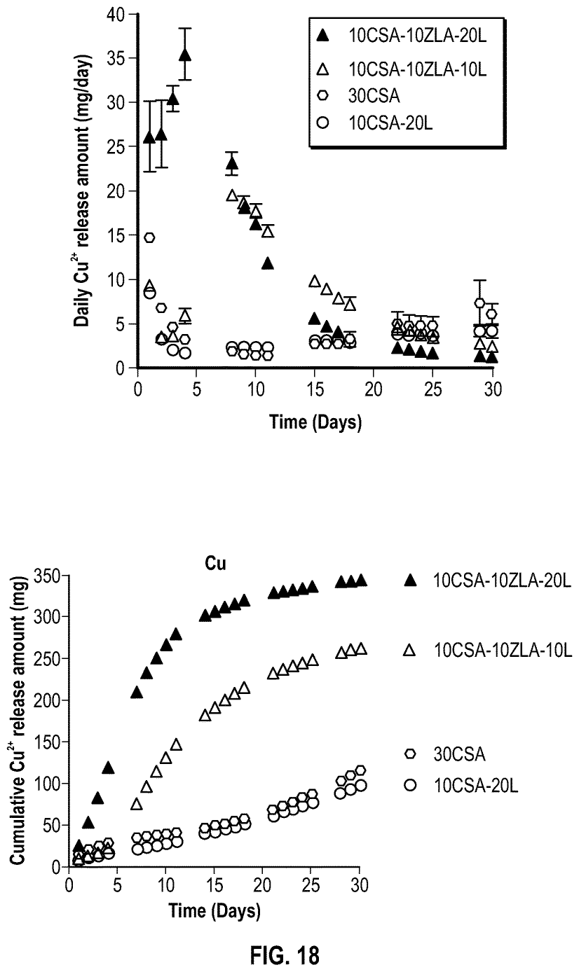

C. trachomatis was incubated with actives ±2% VF and 20% SF for 2 hours at room temperature. The mixtures were serially diluted 1/10 in SPG solution, applied on HeLa cells in triplicate. Images were acquired using the Cytation-5 instrument and the inclusion count was analyzed using the Gen5 software in the Cytation-5. Control conditions included HeLa cells challenged with C. trachomatis in the absence of the actives. (FIG. 15).

Triple combination of selected concentrations of CS (~1-3 mM) and zinc salts (~3-13 mM) and DL-LA (~16-80 mM) provided the strongest anti-viral and anti-C. trachomatis activity in the presence of vaginal fluid (VF) and seminal (SF).

LA concentration was higher than in other models as no activity was observed at lower concentrations. The concentration tested (80 mM) was within physiological range and target release. CS and LA significantly inhibited infection in the absence or presence of the fluids with exception of ZL. Fluids had no significant effect on the activity.

### Ring Formulation Studies

API: CSA-CuSO4 (anhydrous), ZLA-zinc lactate (anhydrous), L-DL-lactide, alone or in combination (CZL which is referred here to CSA-ZLA-L)

The present disclosure provides formulation development and testing of a new non-hormonal multipurpose IVR technology for the sustained release of three actives—copper sulphate anhydrous (CSA), zinc lactate (anhydrous) (ZLA), and DL-lactide (L; hydrolyses to lactic acid, LA) (aka. ‘CZL’ ring).

### Methods for Preparing Matrix-Type LSR-4350 IVRs

Matrix-type silicone (Silbione® LSR-4350 vaginal rings (Table 12) were manufactured using custom ring molds (outer diameter 57.6 mm, cross sectional diameter 7.9 mm) fitted to an electrically-heated, laboratory-scale injection molding machine.

TABLE 12
Compositions of LSR-4350 IVR formulations

| Vaginal ring formulation | |
|---|---|
| (all loadings in % w/w) | Silicone Part A and B and APIs required per batch size |
| Placebo ring - Blank ring | LSR-4350: 240 g. |
| | Total batch size: 240 g |
| 30CSA | LSR-4350: 105 g, CSA: 90 g. |
| | Total batch size: 300 g |
| 30L | LSR-4350: 105 g, L: 90 g. |
| | Total batch size: 300 g |
| 10CSA-20L | LSR-4350: 105 g, CSA: 30 g, L: 60 g. |
| | Total batch size: 300 g |
| 10CSA-10ZLA-10L | LSR-4350: 105 g, CSA: 30 g, L: 30 g, ZLA: 30 g. |
| | Total batch size: 300 g |
| 10CSA-10ZLA-20L | LSR-4350: 90 g, CSA: 30 g, L: 60 g, ZLA: 30 g. |
| | Total batch size: 300 g |

All ring formulation were cured at 115° C. for 3 minutes.

Each ring was packed in a foil pouch and sealed within 2 minutes post ring demoulding.

### In Vitro Release Testing (IVRT)

In vitro release testing of (three rings per formulation) was performed over 30 days. Each ring was placed into an individually labelled 100 mL plastic container. 20 mL of MilliQ ultrapure water was added to each container. Containers were capped and placed in a SciQuip Incu-Shake FL16-2 orbital shaking incubator (37° C., 60 rpm, 25 mm orbital throw). The IVR samples were analysed for lactide and lactic acid using High-Performance Liquid Chromatography (HPLC) and copper and zinc ions using Inductively Coupled Plasma-Optical Emission Spectrometer (ICP-OES). After 24 h±15 min containers will be removed from the incubator and shaken for ~10 seconds before 1-2 mL of the release media is collected for HPLC analysis and 10 mL for ICP-OES analysis (or as required). Then, the release media was completely replaced with 20 mL of fresh MilliQ water (except for weekends when 60 mL was added to maintain release rates), and the capped containers returned to the incubator.

### HPLC Analysis

IVR samples were analysed for DL-lactide and lactic acid using a Waters HPLC system (Waters Limited, Ireland). Briefly, 50 μL of each in vitro release sample was injected onto a Thermo Scientific BDS Hypersil™ C18 column (150×4.46 mm, 3 μm particle size) maintained at 35° C. and fitted with a guard column. Isocratic elution was performed at 1.3 mL/min using a mobile phase of 4% v/v acetonitrile and 96% v/v potassium phosphate buffer (7.7 mM; pH 3.0) with a run time of 5 min. Lactic acid and DL-Lactide were detected at a wavelength of 210 nm after 1.8 min and 3.7 min, respectively.

### ICP-OES Analysis

In vitro release samples were analysed for copper and zinc ions using an ICP-OES (PerkinElmer Avio® 220 Max ICP-OES, UK) equipped with a glass MEINHARD® Type K1 nebulizer and a baffled cyclonic spray chamber (standard).

### Results

### Ring Properties

The dimension of each IVR was ~57.4×~7.3 mm, and the weight was within 7.8-9.3 g. The mechanical properties were similar to the marketed VR products (20 mm compression force, Shore M hardness values and percentage of elongation at break ranged 0.8-4.3 N, 41-67, 248%-564% respectively).

### In Vitro Release of Lactide (LT) and Lactic Acid (LA)

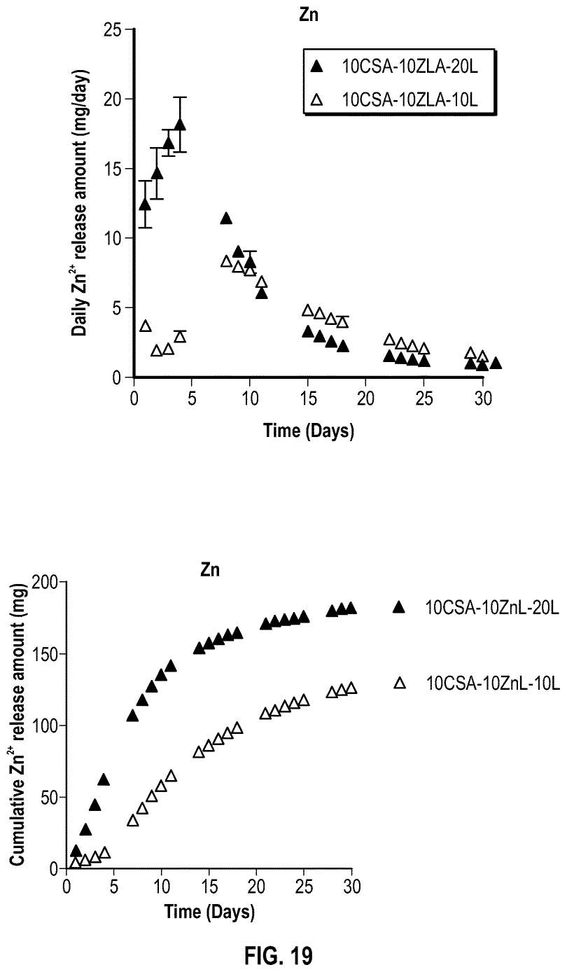

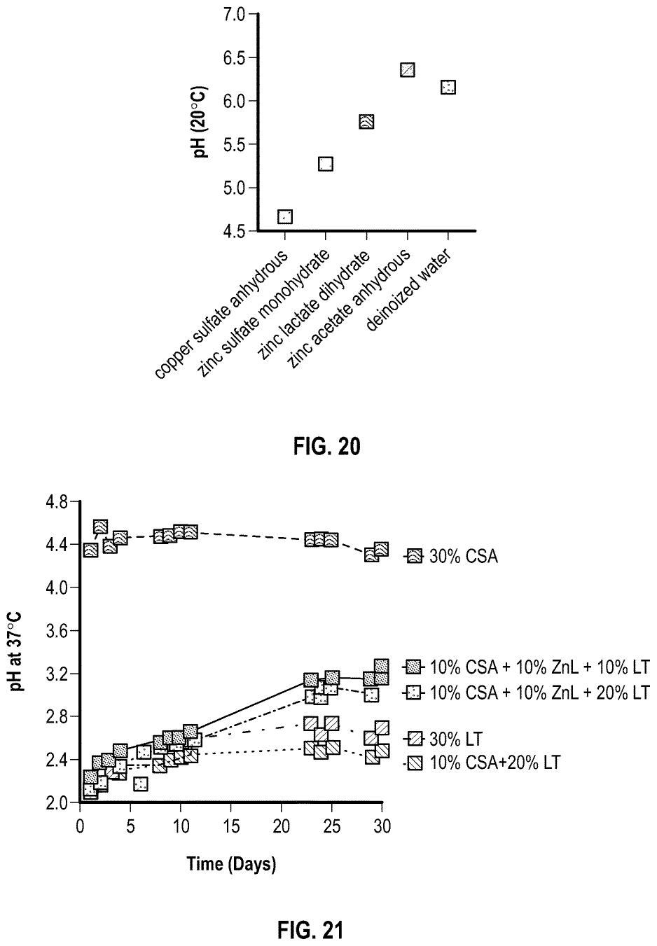

FIG. 16 shows the daily and cumulative release of lactide (LT) and FIG. 17 shows the daily and cumulative release of lactic acid (LA) from the IVRs into deionized water over 30 days.

All lactide loaded IVRs in deionised water were releasing LT and LT in the form of LA. As lactide is intended to readily hydrolyse to lactic acid (both in vitro and in vivo), and thus, it is more informative to assess and measure lactic acid release.

A burst release of lactide over the first five days is observed, which is followed by little or no release thereafter. The mean day one LT burst release in 20 mL of deionised water at 37° C. was more than 122 mg. 10CSA-10ZLA-20L rings released 248.5 mg of LT and 48.4 mg of LA in day one, which is almost two times higher than LT and LA released from 10CSA-10ZLA-10L rings in that day.

Toward the end of second week, the 10CSA-10ZLA-20L IVR daily LA release (86.4 mg/day) was almost two times higher LA released (48.9 mg/day) from 10CSA-10ZLA-10L rings, while its was 27.7 mg/day for 30L rings, and 34.3 mg/day for 10CSA-20L rings.

In week 4, the mean LA daily release from 30L rings and 10CSA-20L rings was ranging from 22.5 to 23.5 mg/day, but LA daily release was dropped to the range of 11.0 mg/day for 10CSA-10ZLA-10L rings and 10CSA-10ZLA-20L rings. This shows that these three actives loaded ring formulations were releasing LT and LA in a relatively higher rate than other formulations over the first two weeks of the 30-day study.

Sustained and continuous release of lactic acid (12-105 mg/day) was observed for triple-active IVRs (1° C. SA-10ZLA-20L) over 30 days.

TABLE 13
Summary of LT and LA daily release data (3 rings per formulation, each ring 20 mL of in deionised
water at 37° C./60 RPM) over 30 days. Each value represents mean ± standard deviation (sd)

| | | Day 1 | Week 1 | | Week 2 | | Week 3 | | Week 4 | |
|---|---|---|---|---|---|---|---|---|---|---|
| LSR-4350 ring | | Mean | Mean | | Mean | | Mean | | Mean | |
| formulations | API | (mg/day) | (mg/day) | SD | (mg/day) | SD | (mg/day) | SD | (mg/day) | SD |
| 30L | LT | 220.1 | 111.7 | 73.5 | 3.8 | 0.8 | 1.1 | 0.3 | 0.4 | 0.1 |
| | LA | 23.8 | 16.3 | 4.8 | 27.7 | 2.5 | 26.6 | 2.5 | 22.5 | 2.4 |
| 10CSA-20L | LT | 198.4 | 81.2 | 77.5 | 0.1 | 0.1 | 0.1 | 0.0 | 0.0 | 0.0 |
| | LA | 25.4 | 34.0 | 11.1 | 34.3 | 4.8 | 27.1 | 4.0 | 23.5 | 3.9 |
| 10CSA-10ZLA-10L | LT | 122.8 | 54.0 | 43.6 | 3.0 | 1.5 | 0.3 | 0.1 | 0.0 | 0.0 |
| | LA | 22.1 | 21.6 | 2.7 | 48.9 | 3.2 | 23.6 | 3.7 | 10.9 | 1.4 |
| 10CSA-10ZLA-20L | LT | 248.5 | 133.1 | 72.5 | 7.9 | 6.1 | 0.4 | 0.1 | 0.0 | 0.0 |
| | LA | 48.4 | 53.4 | 10.8 | 86.4 | 16.5 | 28.5 | 5.7 | 11.7 | 1.8 |

Over the 30-day study, the total LT cumulative release percent was higher (almost 100% L released) from the three actives leaded ring formulations (regardless the LT loading; 10% or 20% w/w) than 10CSA-20LT rings (62.9%), which was still higher than from 30L rings (44.7%).

### In Vitro Release of Zinc Ion and Copper Ion

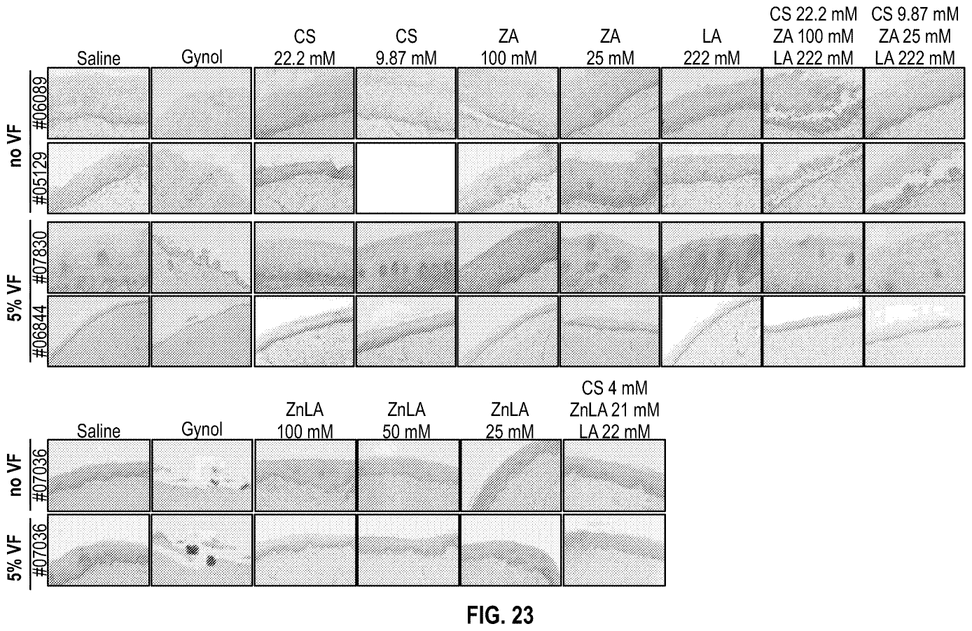

FIG. 19 shows the daily and cumulative release of zinc ion from the IVRs into deionized water over 30 days.

For zinc ion release, no characteristic permeation-controlled Day 1 burst release was observed on the 10CSA-10ZLA-10L (3.7 mg) and 10CSA-10ZLA-20L (12.5 mg) formulations. The 10CSA-10ZLA-20L formulation (the 40% w/w actives-loaded ring) showed a Day 1 zinc ion release of three times higher than the 10CSA-10ZLA-10L (the 30% w/w actives-loaded ring), which was boosted by extra 10% w/w DL-lactide in the ring. The mean Week 1 (Day 2-4) zinc ion release of the 10CSA-10ZLA-20L formulation (16.6 mg) was higher than that of the 10CSA-10ZLA-10L formulation (2.3 mg). The following daily release of zinc ion after Week 1 from both the 10CSA-10ZLA-20L formulation and the 10CSA-10ZLA-10L formulation decreased with time. The huge difference in the cumulative release of zinc ion from the two formulations is mainly from the first week (Week 1) release. By comparison, the highest release rate appeared in Week 1 for the 10CSA-10ZLA-20L formulation, and in Week 2 for the 10CSA-10ZLA-10L formulation. There was a lag effect in the Week 1 release of the 10CSA-10ZLA-10L formulation.

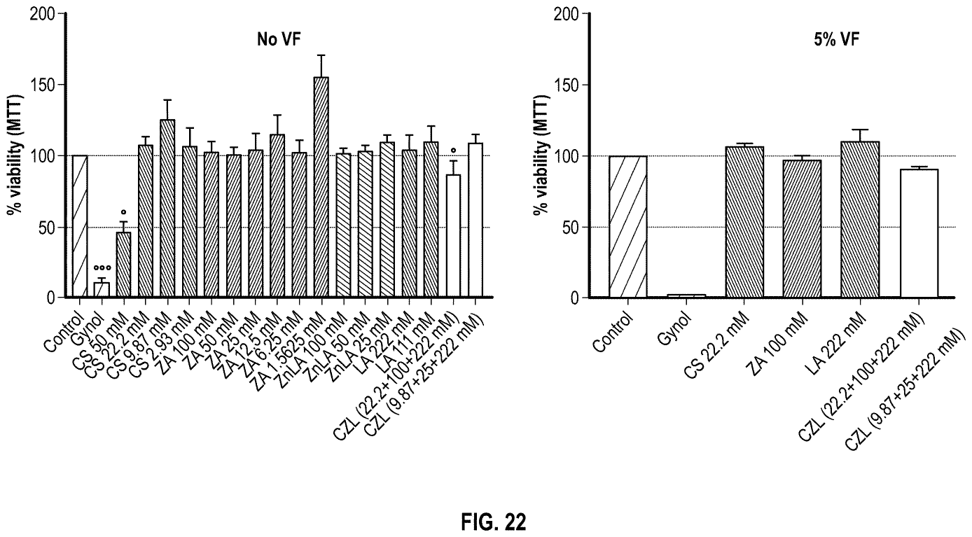

FIG. 18 shows the daily and cumulative release of copper ion from the IVRs into deionized water over 30 days.

For copper ion release, similar profiles of daily release and cumulative release with zinc ion were observed on the 10CSA-10ZLA-10L and 10CSA-10ZLA-20L formulations. As shown in Table 15, in general, daily copper ion release was always higher than zinc ion release for each formulation. 56% of zinc ion and 78% of copper ion released from the 10CSA-10ZLA-10L ring; 78% of zinc ion and 99% of copper ion released from the 10CSA-10ZLA-20L ring. The same drug loading of CSA and ZLA theoretically presents the same and homogeneous distribution in the silicone elastomer ring. The higher release of copper ion is likely due to a faster dissolution rate in the release medium (deionised water) since the reported water solubilities of CSA and ZLA are 201 and 48.2 g/L (20° C.) in water, respectively.

For 30CSA and 10CSA-20L formulations, both are 30% w/w actives-loaded rings. Both showed a burst Day 1 copper ion release in week 1-14.7 and 8.5 mg, respectively. The copper ion release rate of 30CSA decreased in Week 2 and then increased in Week 3 and 4; while 10CSA-20L showed a relatively consistent copper ion release rate in Week 2 and then an increase in Week 3 and 4. For both formulations, the highest release rate appeared in Week 4. It seems that the lag time of 30CSA and 10CSA-20L is longer than 10CSA-10ZLA-10L and then 10CSA-10ZLA-20L ring in terms of the order of the appearance of the highest release rate over 30 days. Compared to the 10CSA-10ZLA-10L ring, only 10% and 30% copper ion released from the 30CSA and 10CSA-20L rings, respectively.

Sustained and continuous release of copper and zinc ions (2-31 and 1-17 mg/day, respectively) was observed for triple-active IVRs (1° C. SA-10ZLA-20L) over 30 days.

TABLE 14
Mean copper ion and zinc ion release on Day 1, Week 1(Day 2-4), Week 2 (Day
8-11), Week 3 (Day 15-18), Week 4 (Day 22-25), mean 30-day cumulative release
and mean percentage of release of vaginal ring formulations in X01 study

| | | | | | | | |
|---|---|---|---|---|---|---|---|
| | Day 1 | Week 1 | Week 2 | Week 3 | Week 4 | 30-day cumulative | Cumulative |
| Ring Formulation | (mg) | (mg/day) | (mg/day) | (mg/day) | (mg/day) | release (mg) | release % |

Mean zinc ion release values

| | | | | | | | |
|---|---|---|---|---|---|---|---|
| 10CSA-10ZLA-10L | 3.7 | 2.3 | 7.7 | 4.4 | 2.4 | 126 | 56 |
| 10CSA-10ZLA-20L | 12.5 | 16.6 | 8.7 | 2.8 | 1.4 | 182 | 78 |

Mean copper ion release values

| | | | | | | | |
|---|---|---|---|---|---|---|---|
| 30CSA | 14.7 | 4.9 | 1.6 | 2.9 | 4.8 | 97 | 10 |
| 10CSA-20L | 8.5 | 2.3 | 2.3 | 3.1 | 3.8 | 116 | 30 |
| 10CSA-10ZLA-10L | 9.3 | 4.4 | 17.8 | 8.5 | 4.0 | 262 | 78 |
| 10CSA-10ZLA-20L | 26.1 | 30.8 | 17.3 | 4.5 | 2.0 | 343 | 99 |

### In Vitro Release PH Profile

As low pH in vivo is anticipated to be a major contributor to immobilisation of sperm, the effect of the API on medium pH was studied.

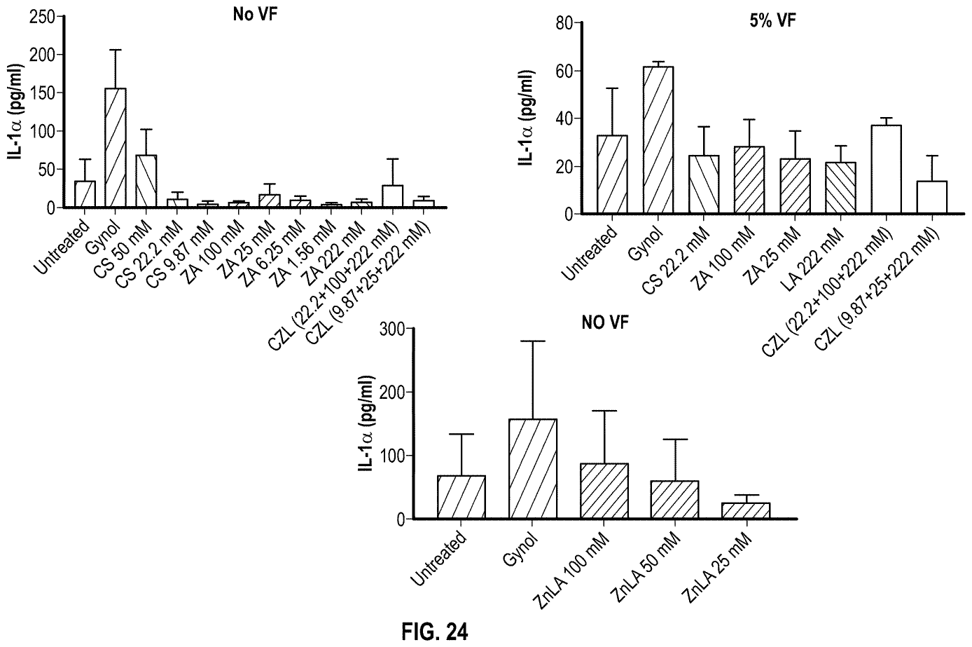

FIG. 20 shows the pH of 1 mg/mL solution of various salts, including copper sulfate anhydrous, zinc sulfate monohydrate, zinc lactate dihydrate, and zinc acetate anhydrous.

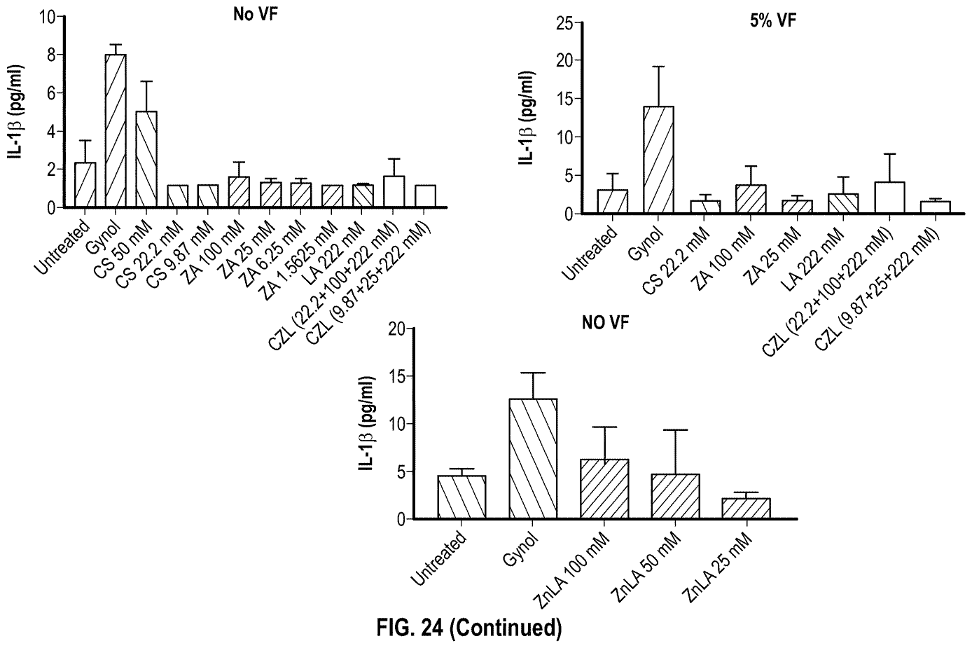

FIG. 21 shows the pH of the release medium during the 30-day in vitro release study for five IVRs (30CSA, 1° C. SA-10ZLA-10L, 1° C. SA-10ZLA-20L, 30L, and 10CSA-20L). The pH of all the release medium during the 30-day for 10CSA-10ZLA-10L, 10CSA-10ZLA-20L, 30L, and 10CSA-20L was maintained below 4.6. The reduction in pH for these rings is thought to be consistent with the release of lactide and its rapid hydrolysis to form lactic acid. The copper and zinc salts further modulate the medium pH, in accordance with the well-established Lewis acid characteristics of these metal ions and the acid-base behaviour of the counter ions.

Over 30 days release in deionised water, the pH range was between 2.1 to 3.3 for lactide loaded IVRs, which was consistent with that lactide can hydrolyse in the presence of water into lactic acid, while the mean pH value for the 30CSA IVR was 4.5±0.1 (Table 15). It was noted that the healthy vaginal pH should be between 3.8-4.5. The pH of deionised water should be between 5.5-6.0.

TABLE 15
Summary of pH data during the APIs loaded LSR-4350
rings (3 rings per formulation, each ring 20 mL of
in deionised water at 37° C./60 RPM) over 30
days. Each value represents mean ± standard deviation (sd)

| Ring Formulation | pH - Day 1 | pH - Day 30 | Mean pH over 30 days |
|---|---|---|---|
| 30CSA | 4.3 (0.1) | 4.4 (0.1) | 4.5 (0.1) |
| 30L | 2.1 (0.1) | 2.7 (0.1) | 2.5 (0.2) |
| 10CSA-20L | 2.1 (0.1) | 2.5 (0.1) | 2.4 (0.1) |
| 10CSA-10ZLA-10L | 2.2 (0.1) | 3.3 (0.1) | 2.7 (0.3) |
| 10CSA-10ZLA-20L | 2.2 (0.1) | 3.2 (0.1) | 2.6 (0.3) |

### Toxicity Assays

APIs: copper sulfate (CS); DL-lactic acid (LA); zinc acetate (ZA) or zinc lactate (ZnLA or ZL); or in combination

### Single Exposure to APIs

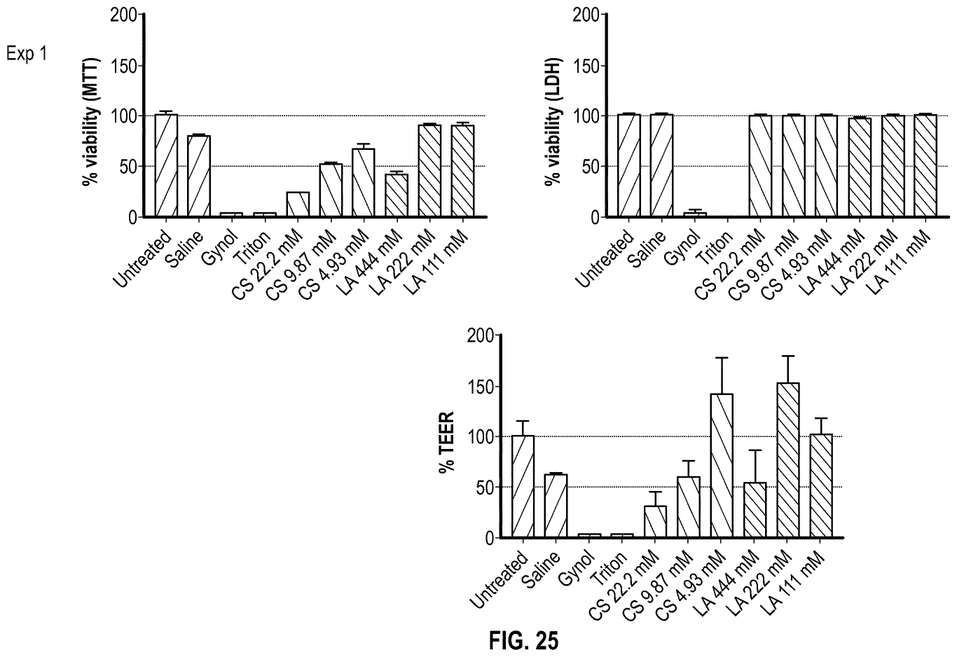

Ectocervical explants were exposed to the APIs [copper sulfate (CS); DL-lactic acid (LA); zinc acetate (ZA) or zinc lactate (ZnLA or ZL); or in combination] for 24 hours (hr) vs. control (saline±5% VF) and Gynol condition. Shown is tissue viability (%) relative to saline control in the absence (n=3-11 experiments) or presence (n=3 experiments) of 5% VF (Mean±SEM). p values represent comparisons between indicated treatment and saline (* <0.05; *** <0.0005). Kruskal-Wallis test with Dunn's multiple comparison) (FIG. 22). Table 16 shows the amount of APIs used in a single treatment.

TABLE 16
Toxicity - Single treatment

| | Non-toxic | Non-toxic | | | |
|---|---|---|---|---|---|
| | concentrations | concentrations | Non-cidal | Anti-Sperm concentrations | |
| | in ectocervix | in VEC-100 | concentrations | | Sperm- |
| | MTT and | model * | in | | mucus |
| | histology 24 | TEER and | lactobacillus | Total motility | penetration |
| API | hr | LDH 24 h | MCC 30 min | (EC₉₀) | (EC₉₀) |
| CS (mM) | 22 | 10 | 10 | 4 | 0.15 |
| ZL (mM) | 100 | 100 | 100 | 22 | 5 |
| LA (mM) | 222 | 222 | 222 | 21 | 1.5 |

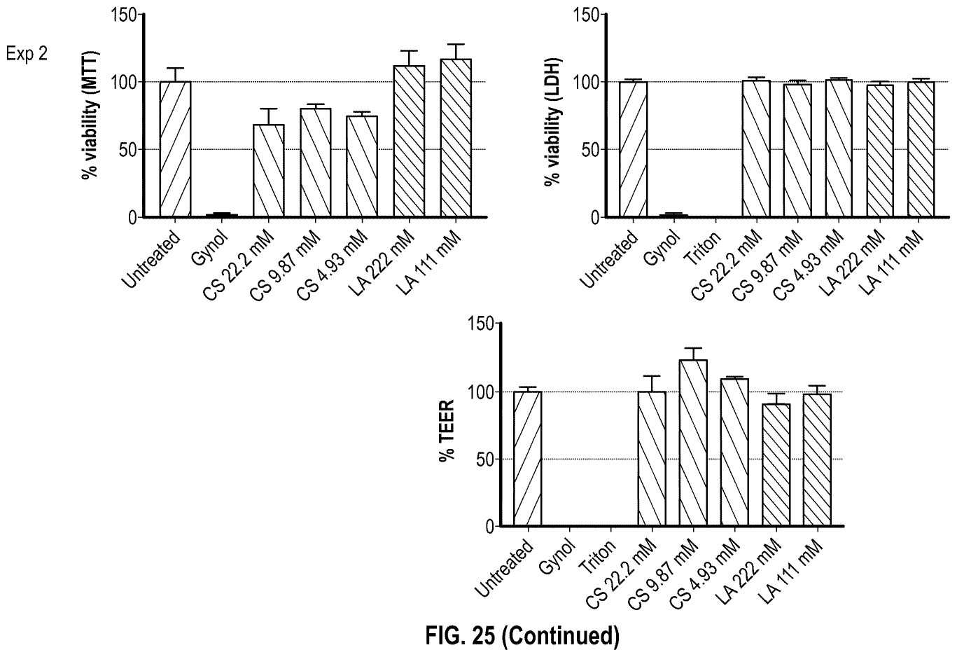

In FIG. 23, ectocervical explants were incubated with the selected concentrations of the APIs for 24 hr in the presence or absence of 5% VF. Explants were fixed, paraffin embedded, sectioned and stained with H&E.

### Inflammatory Cytokines Concentrations Following Single Exposure of Ectocervical Explants to the APIs

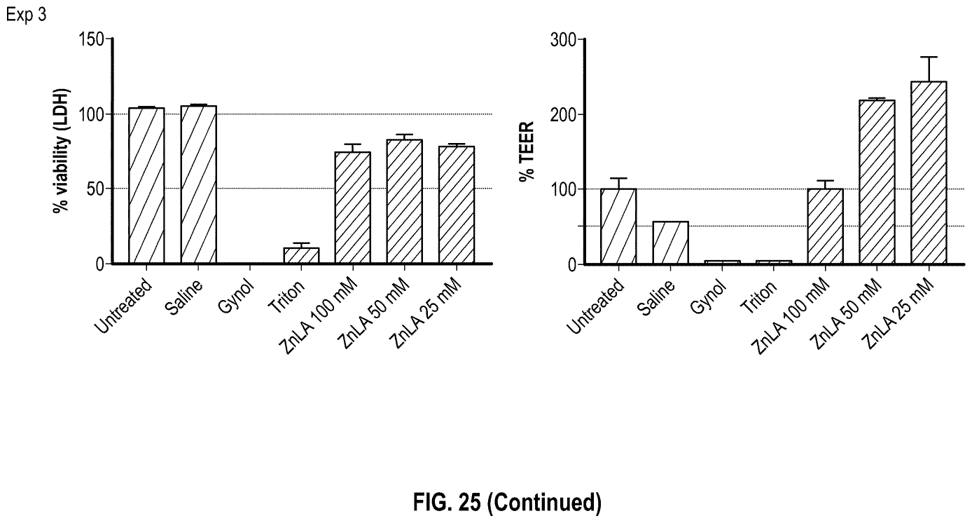

Ectocervical explants were exposed to the APIs 24 hr in the presence or absence of 5% VF. Supernatants were collected and analyzed for pro-inflammatory cytokines. Shown are Mean±SEM (n=3-6 experiments) of IL-la and IL-1P (FIG. 24).

### Viability and TEER in VEC-100 Tissue Following Single Exposure to the APIs

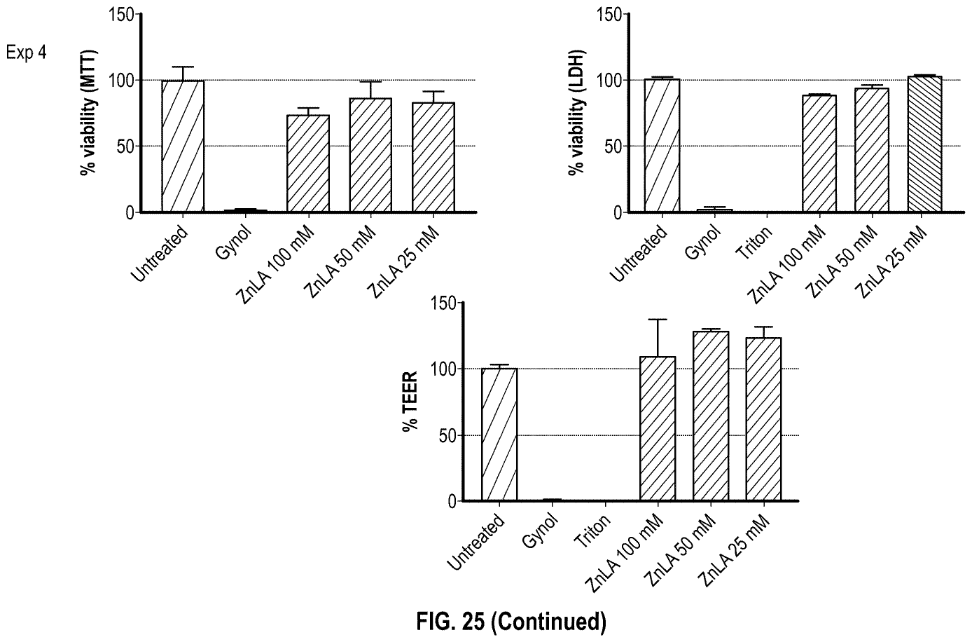

VEC-100 tissues were incubated with APIs for 24 hr. Tissue viability was analyzed by MTT and LDH assay and TEER measurements were recorded. Untreated, Gynol and Triton conditions were included as controls. Data from 4 individual experiments are presented (FIG. 25).

Table 17 shows the summary of the findings for indicated concentrations of the APIs after single exposure.

TABLE 17
Safety of the single API in ectocervix
and VEC-100 following single exposure

| | Ectocervix: single exposure | | VEC-100: Single exposure |
|---|---|---|---|
| | (MTT, Histology, CC) 24 h | | (MTT, TEER and LDH) 24 h |
| Actives | No VF | 5% VF | No VF |
| Gynol | Tox | Tox | Tox |
| CS 22.2 mM | No Tox | No Tox | Tox |
| CS 9.87 mM | No Tox | No Tox | No Tox |
| CS 4.93 mM | No Tox | No Tox | No Tox |
| ZA 100 mM | No Tox | No Tox | No Tox |
| ZL 100 mM | No Tox | No Tox | No Tox |
| ZL 50 mM | No Tox | No Tox | No Tox |
| ZL 25 mM | No Tox | No Tox | No Tox |
| LA 222 mM | No Tox | No Tox | No Tox |
| LA 111 mM | No Tox | No Tox | No Tox |

Exposure to CS (up to 10 mM), zinc salts (up to 100 mM), and LA (up to 222 mM) alone did not induce toxicity. No histopathological changes, decrease in tissue viability (MTT, LDH), decrease in TEER or increase in cytokine concentrations was noted after 24 h exposure to single actives. Tripe actives combinations CS 10 mM, ZA 25 mM and LA 222 mM in saline results in epithelial damage in ectocervical explants. However, no damage was detected in the presence of 5% VF.

### Multiple Exposure to APIs

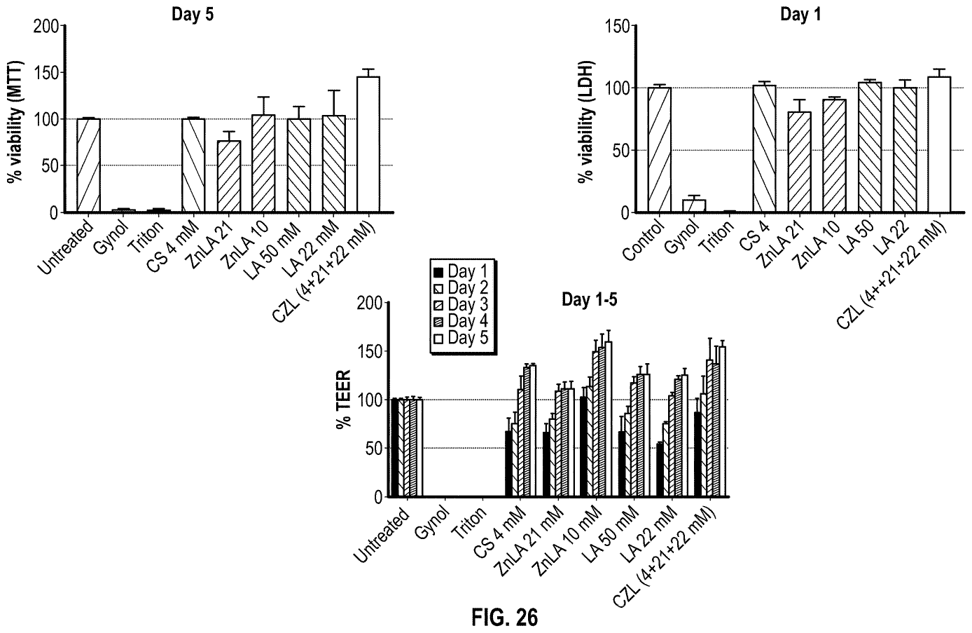

VEC-100 tissues were incubated with selected concentrations (showing synergy in anti-sperm assays: CS 4 mM, ZL 21 mM, LA: 22 mM)) of the APIs (single and combinations) for 1 hr daily for 5 days, rinsed in PBS and further incubated for 23 hours. Single experiment is shown. Tissue viability and TEER were analyzed on indicated days (Mean±SEM of replicates) as shown in FIG. 26. Table 18 illustrates that indicated concentrations are safe and have anti-sperm activity.

TABLE 18
Toxicity - Multiple Treatments

| | Non-toxic | Non- toxic | | |
|---|---|---|---|---|
| | concentrations in | concentrations based | Anti-Sperm concentrations | |
| | VEC-100 model * | on rat vaginal | | Sperm-mucus |
| | TEER and LDH 5 | irritation study | Total motility | penetration |
| API (mM) | daily exposures | 10 days exposure | (EC₉₀) | (EC₉₀) |
| CS (mM) | 4 | 22 | 4 | 0.15 |
| ZL (mM) | 21 | 50 | 22 | 5 |
| LA (mM) | 22 | 200 | 21 | 1.5 |

Table 19 shows the summary of the findings for indicated concentrations of the APIs after multiple exposure.

TABLE 19
Safety of the APIs in ectocervix and
VEC-100 following repeated exposure

| | |
|---|---|
| | VEC-100 single exposure (MTT, TEER |
| | and LDH) day 1-5 |
| | 24 h |
| Actives* | No VF |

| | | | |
|---|---|---|---|
| Gymol | Tox | Tox | Tox |
| Triton | Tox | Tox | Tox |
| CS 4 mM | No Tox | No Tox | No Tox |
| ZnLA 21 mM | No Tox | No Tox | No Tox |
| ZnLA 10 mM | No Tox | No Tox | No Tox |
| LA 22 mM | No Tox | No Tox | No Tox |
| CS 4 mM | No Tox | No Tox | No Tox |
| ZnLA 21 mM | | | |
| LA 22 mM | | | |

Individual activities and their combinations (CS 4 mM, ZL 21 mM, LA 22 mM) did not decrease tissue viability (MTT, LDH) and TEER.

1. An intravaginal ring comprising a therapeutically effective amount of a non-hormonal active agent dispersed in an elastomer, wherein the non-hormonal active agent comprises a lactide component, and at least one further component selected from a copper component and a zinc component.

2-3. (canceled)

4. The intravaginal ring of claim 1, wherein the non-hormonal active agent comprises a lactide component, a copper component, and a zinc component.

5. The intravaginal ring of claim 1, wherein the copper component is metallic copper, copper oxide, a copper salt or a copper ion-ligand complex.

6. The intravaginal ring of claim 1, wherein the zinc component is metallic zinc, zinc oxide or a zinc salt.

7. The intravaginal ring of claim 1, wherein the lactide component is D-lactide, L-lactide, DL-lactide or lactic acid.

8. The intravaginal ring of claim 1, wherein the elastomer is selected from silicone, polyethylene vinyl acetate copolymer (EVA), styrene-butadiene-styrene block copolymer, polyphosphazene, poly(isoprene), poly(isobutylene), polybutadiene, polyurethane, a nitrile rubber, a neoprene rubber, and a combination thereof.

9. The intravaginal ring of claim 1, wherein the elastomer is included in an amount of about 50% to about 99% by a total weight of the intravaginal ring.

10. The intravaginal ring of claim 1, wherein the intravaginal ring is in a form of a matrix, and the elastomer is silicone.

11. The intravaginal ring of claim 1, wherein the intravaginal ring is in a form of an exposed-core, and the elastomer is polyethylene vinyl acetate or polyurethane.

12. The intravaginal ring of claim 5, wherein the copper salt is anhydrous copper sulfate or copper sulfate hydrate.

13. The intravaginal ring of claim 6, wherein the zinc salt is zinc acetate, zinc formate, zinc lactate, zinc chloride, zinc sulfate, zinc iodide, zinc citrate, or zinc orotate, each of which is in an anhydrous or a hydrate form.

14. The intravaginal ring of claim 1, wherein the non-hormonal active agent is included in an amount of about 5 wt % to about 50 wt %, based on a total amount of the intravaginal ring.

15. The intravaginal ring of claim 4, having copper ions, zinc ions and lactic acid released in a molar ratio of 1:1:1 to 1:6:6 after an initial first day period.

16. The intravaginal ring of claim 4, having a release of copper ions at a rate of 2 mg/day to 31 mg/day during a 30-day period of use.

17. The intravaginal ring of claim 4, having a release of zinc ions at a rate of 1 mg/day to 17 mg/day during a 30-day period of use.

18. The intravaginal ring of claim 4, having a release of lactic acid at a rate of 12 mg/day to 105 mg/day during a 30-day period of use.

19. A method for preventing unintended pregnancy or preventing and/or treating a disease caused by bacterial or viral infection, comprising intravaginally administering the intravaginal ring of claim 1 to a female subject in need thereof.

20. The method according to claim 19, wherein said disease is a sexually transmitted infection.
